(19) United States
(12) Patent Application Publication
(10) Pub. No.: US 2020/0306377 A1
(43) Pub. Date: Oct. 1, 2020

(54) METHODS FOR ENHANCING 5-AMINOLEVULINIC ACID-BASED MEDICAL IMAGING AND PHOTOTHERAPY

(71) Applicant: Nanoco Technologies Ltd., Manchester (GB)

(72) Inventors: Imad NAASANI, Manchester (GB); Mark SAUNDERS, Manchester (GB)

(21) Appl. No.: 16/754,625

(22) PCT Filed: Oct. 18, 2018
(86) PCT No.: PCT/GB2018/053019

§ 371 Date: Apr. 8, 2020

Related U.S. Application Data
(60) Provisional application No. 62/573,971, filed on Oct. 18, 2017.

(51) Int. Cl. A61K 41/00; A61K 47/64; A61P 35/00

(52) U.S. Cl. CPC A61K 41/0061; A61P 35/00; A61K 47/64

(57) ABSTRACT

The present disclosure relates to quantum dot nanoparticles conjugated to 5-Aminolevulinic acid or esters thereof and their uses in conjunction with additional free, non-endogenous 5-Aminolevulinic acid or esters thereof.

### REFERENCE TO RELATED APPLICATIONS

This application claims priority based on U.S. Provisional Application Ser. No. 62/573,971 filed Oct. 18, 2017, which is incorporated herein by reference in its entirety.

### FIELD OF THE INVENTION

Embodiments disclosed herein relate to quantum dot nanoparticles conjugated to 5-Aminolevulinic acid (5-ALA), and in particular methods for enhancing 5-Aminolevulinic acid (5-ALA) based medical imaging and phototherapy with the use of quantum dot nanoparticles (QDs).

### BACKGROUND OF THE INVENTION

Photodynamic therapy (PDT) is a treatment that uses a photosensitive drug, called a photosensitizer (PS), along with light to kill undesirable cells including precancerous and cancer cells. The drugs only work after they have been activated by light. Upon irradiation with appropriate light, the photosensitizer produces reactive oxygen species (ROS) for the destruction of the undesired tissue such as, in particular, neoplastic tissue.

5-Aminolevulinic acid (5-ALA) is an approved PS for PDT and is widely used. It is also used as a marker in fluorescence guided surgery of certain inflammatory tissues and cancers including gliomas, bladder cancer and melanomas. 5-ALA is a prodrug, and once internalized into tumor cells, 5-ALA undergoes conversion to the natural photosensitizer protoporphyrin IX (PpIX). In contrast to exogenously administered PSs, such as the porphyrin drug porfimer sodium (PHOTOFRIN®), which can result in photosensitivity reactions in the skin and eye with exposure to ambient light, 5-ALA is a photodynamically inactive, non-selective and non-toxic compound that is intracellularly metabolized to the photodynamically active and fluorescent PpIX. Subsequent illumination of the tumor site with red light activates PpIX, triggers the oxidative damage and induces cytotoxicity. The photodynamically active and fluorescent PpIX can also be used as a marker in the fluorescence guided surgery of certain inflammatory tissues and most cancers like gliomas, bladder cancer and melanomas.

However, 5-ALA is a polar molecule. The zwitterionic nature and hydrophilicity of 5-ALA greatly limit its penetration through tissues, such as intact skin, nodular skin lesions and through cell membranes, leading to a slow cellular uptake and an inconsistent accumulation of PpIX in tumor cells. Thus, 5-ALA penetration through the cell membrane and targeted delivery to tumor cells are challenges in improving the efficacy and specificity of PDT. These challenges render 5-ALA treatment unsatisfactory for use with certain types of tumors, such as, for example, brain tumors. Accordingly, there is a need to enhance the efficacy of 5-ALA treatment.

### SUMMARY OF THE INVENTION

Embodiments disclosed describe methods for enhancing the performance of 5-ALA in which quantum dot nanoparticles (QDs) are conjugated to 5-ALA or 5-ALA esters (for example, tethered to the QD surface) and are co-administered with free 5-ALA or 5-ALA ester.

In certain embodiments the 5-ALA ester is selected from 5-ALA hexyl ester, 5-ALA methyl ester, aliphatic alcohol 5-ALA esters, glycoside 5-ALA esters including α-glucose, α-mannose, or β-galactose esters of 5-ALA, and alkyl esters of 5-ALA.

Surprisingly, the combination of these two 5-ALA types (both conjugated and free) shows a mutually synergistic effect with enhanced emission from the formed PpIX molecules resulting in enhanced performance of 5-ALA treatment.

Embodiments disclosed include quantum dot nanoparticles, wherein each QD is bonded (e.g., covalently bonded or physically bonded (by ion pairing or van der Waals interactions) to 5-ALA, e.g., by aliphatic chains, π-π stacking, π interactions, an amide, ester, thioester, or thiol anchoring group directly on an inorganic surface of the QD, or on an organic corona layer that is used to render the QD water soluble and biocompatible. The water soluble QD in certain embodiments include a core of one semiconductor material and at least one shell of a different semiconductor material in some embodiments while in other embodiments the water soluble QD includes an alloyed semiconductor material having a bandgap value that increases outwardly by compositionally graded alloying. Such embodiments are useful, for example, for the visualization and treatment of cancer, both ex vivo and in vivo.

In one embodiment, each QD is conjugated to 5-ALA or an ester thereof that may be activated by a light source.

In one embodiment, each QD described herein is covalently linked to 5-ALA or an ester thereof via an amide bond.

In one embodiment, each QD comprises: a core semiconductor material, and an outer layer, wherein the outer layer comprises a corona of organic coating (a functionalization organic coating) to render the particles water soluble and bio compatible, and 5-ALA or an ester thereof. In one embodiment, each QD comprises one or more shells of semiconductor material, the outer shell comprising an outer layer, wherein the outer layer comprises a corona of organic coating (a functionalization organic coating) to render the particles water soluble and bio compatible, and 5-ALA or an ester thereof.

In one embodiment, each QD comprises: an alloyed quantum dot and 5-ALA or an ester thereof. In one embodiment, each QD comprises: a doped quantum dot and 5-ALA or an ester thereof. In one embodiment of any of the QD described herein, the nanoparticle comprises a II-VI material, a III-V material, or I-III-IV material, or any alloy or doped derivative thereof.

In one embodiment, any of the QD described herein are associated with an emission spectrum ranging from about 350 nm to about 1000 nm and further from about 450 nm to about 800 nm.

In an additional embodiment, any of the QD described herein may further comprise a cellular uptake enhancer, a tissue penetration enhancer, or any combination thereof. Examples of cellular uptake enhancers include, for example, trans-activating transcriptional activators (TAT), Arg-Gly-Asp (RGD) tri-peptides, linear and cyclic peptides including the RGD motif, or poly arginine peptides. Examples of tissue penetration enhancers include saponins, cationic lipids, and Streptolysin O (SLO).

In other embodiments, at least one target specific ligand is conjugated to a water soluble non-toxic QD together with 5-ALA or 5-ALA ester conjugation. Examples of targets to which the target specific ligands are specific include EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand.

In another embodiment, a method of inducing cell death is provided. In another embodiment, a method of inducing cell death and imaging affected tissues is provided.

In another embodiment, a method of visualizing and treating tumors (both malignant and benign), inflammatory tissue, and/or undesired cells is provided. In additional embodiments, the tumor is soft or solid. In addition, the method of visualizing tumors and/or inflammatory tissue can be used for intraoperative imaging and fluorescence guided surgery of the tumors and/or inflammatory tissue. Inflammatory tissues may include, for example, arthritis, Crohn's disease, Inflammatory Bowel Disease, psoriasis, acne, multiple sclerosis, Alzheimer, Parkinson, or any other disease or condition that has a tendency of increased PpIX synthesis. For example, the combination of 5-ALA-QD or an ester thereof and free 5-ALA or an ester thereof is used to detect atherosclerosis plaques, atheromatous lesions and stenosis levels, particularly those of the carotid artery. The combination of 5-ALA-QD and free 5-ALA is used to treat atherosclerosis plaques, atheromatous lesions and stenosis levels, particularly those of the carotid artery.

In another embodiment, a method of visualizing and treating circulating cells in blood or body fluids is provided.

In one embodiment, any of the methods described herein comprises: i) contacting QD conjugates (e.g., a plurality or a panel of QD conjugates) according to any of the embodiments described herein with a cell, tumor or unwanted tissue, and (ii) contacting free 5-ALA or a derivative thereof with the cell.

In an additional aspect of the embodiment, a polymerizable ligand is affixed to the 5-ALA-QD or derivative of 5-ALA such as a 5-ALA ester wherein the ligand is polymerized by excitation of the quantum dot nanoparticles with an energy source (e.g., a light source, such as a UV or visible light source). In certain embodiments the 5-ALA ester is selected from 5-ALA hexyl ester, 5-ALA methyl ester, aliphatic alcohol 5-ALA esters, glycoside 5-ALA esters including α-glucose, α-mannose, or β-galactose esters of 5-ALA, and alkyl esters of 5-ALA.

In one embodiment of any of the methods described herein, the QD-5-ALA conjugates are excited using multi-photon excitation (e.g., a two-photon excitation). In such an embodiment, the combined energy of two or more light beams is used to excite a particular QD-5-ALA conjugates.

In one embodiment, any of the methods described herein are performed in bodily fluids (e.g., blood, pancreatic juice, plasma, fine needle aspirate) and/or tissues samples in vivo via co-administration of the 5-ALA-nanoparticle conjugates together with free 5-ALA into living tissue.

In one embodiment, any of the methods described herein are performed in bodily fluids and/or tissues samples taken and examined ex vivo. For example, the detection of an emission signal can be performed on biological samples removed and tested ex vivo using fluorescence microscopy, flow cytometry or fluorimeters.

In an embodiment, there is provided a combination of QD-5-ALA or an ester thereof and free 5-ALA or an ester thereof.

In an embodiment, there is provided a pharmaceutical composition comprising a combination of QD-5-ALA or an ester thereof and free 5-ALA or an ester thereof, together with a pharmaceutically acceptable carrier.

In an embodiment, there is provided a combination of QD-5-ALA or an ester thereof and free 5-ALA or an ester thereof, for use as a medicament.

In an embodiment, there is provided a combination of QD-5-ALA or an ester thereof and free 5-ALA or an ester thereof, for use in imaging.

In an embodiment, there is provided a combination of QD-5-ALA or an ester thereof and free 5-ALA or an ester thereof, for use in a method of inducing cell death.

In an embodiment, there is provided a combination of QD-5-ALA or an ester thereof and free 5-ALA or an ester thereof, for use in a method of treating tumors (both malignant and benign), inflammatory tissue, and/or undesired cells, comprising administering a therapeutically effective amount of QD-5-ALA or an ester thereof to a patient in need thereof.

In an embodiment, there is provided a combination of QD-5-ALA or an ester thereof and free 5-ALA or an ester thereof, for use in a method of imaging affected tissue.

In an embodiment, there is provided a combination of QD-5-ALA or an ester thereof and free 5-ALA or an ester thereof, for use in a method of visualizing and treating tumors (both malignant and benign), inflammatory tissue, and/or undesired cells, comprising administering a therapeutically effective amount of QD-5-ALA or an ester thereof to a patient in need thereof.

In an embodiment, there is provided a combination of QD-5-ALA or an ester thereof and free 5-ALA or an ester thereof, for use in a method of visualizing and treating circulating cells in blood or body fluids.

In an embodiment, there is provided a combination of QD-5-ALA or an ester thereof and free 5-ALA or an ester thereof, for use in a method of enhancing intracellular PpIX fluorescence.

In an embodiment, there is provided the use of a combination of QD-5-ALA or an ester thereof and free 5-ALA or an ester thereof, for the manufacture of a medicament for inducing cell death.

In an embodiment, there is provided the use of a combination of QD-5-ALA or an ester thereof and free 5-ALA or an ester thereof, for the manufacture of a medicament for the treatment of tumors (both malignant and benign), inflammatory tissue, and/or undesired cells.

In an embodiment, there is provided the use of a combination of QD-5-ALA or an ester thereof and free 5-ALA or an ester thereof, for the manufacture of a medicament for the imaging of affected tissue.

In an embodiment, there is provided the use of a combination of QD-5-ALA or an ester thereof and free 5-ALA or an ester thereof, for the manufacture of a medicament for the visualization and treatment of tumors (both malignant and benign), inflammatory tissue, and/or undesired cells.

In an embodiment, there is provided the use of a combination of QD-5-ALA or an ester thereof and free 5-ALA or an ester thereof, for the manufacture of a medicament for the visualization and treatment of circulating cells in blood or body fluids.

In an embodiment, there is provided the use of a combination of QD-5-ALA or an ester thereof and free 5-ALA or an ester thereof, for the manufacture of a medicament for the enhancement of intracellular PpIX fluorescence.

In an embodiment, there is provided a kit comprising QD-5-ALA or an ester thereof and instructions for the co-administration with free 5-ALA or an ester thereof. In a further embodiment, there is provided a kit comprising QD-5-ALA or an ester thereof and free 5-ALA or an ester thereof, and, optionally, instructions for treating a patient. In a further embodiment, there is provided a kit comprising free 5-ALA or an ester thereof, and instructions for the co-administration with QD-5-ALA or an ester thereof.

In an embodiment, there is provided quantum dots (QDs) conjugated to 5-Aminolevulinic acid (5-ALA), for use in a method for the enhancement of intracellular PpIX fluorescence comprising:

administering quantum dots (QDs) conjugated to 5-Aminolevulinic acid (5-ALA) or esters of 5-ALA to a tissue;

co-administering free 5-ALA or 5-ALA esters to the tissue;

allowing the QD-5-ALA or QD-5-ALA ester conjugates the co-administered free 5-ALA or 5-ALA esters to be internalized by cells within the tissue and form intracellular PpIX; and

physically exciting the QD-5-ALA or QD-5-ALA ester conjugates to induce PpIX fluorescence.

In an embodiment, there is provided the use of quantum dots (QDs) conjugated to 5-Aminolevulinic acid (5-ALA), for use in the manufacture of a medicament for the enhancement of intracellular PpIX fluorescence comprising:

administering quantum dots (QDs) conjugated to 5-Aminolevulinic acid (5-ALA) or esters of 5-ALA to a tissue;

co-administering free 5-ALA or 5-ALA esters to the tissue;

allowing the QD-5-ALA or QD-5-ALA ester conjugates the co-administered free 5-ALA or 5-ALA esters to be internalized by cells within the tissue and form intracellular PpIX; and

physically exciting the QD-5-ALA or QD-5-ALA ester conjugates to induce PpIX fluorescence.

In an embodiment, there is provided quantum dots (QDs) conjugated to 5-Aminolevulinic acid (5-ALA), for use in a method of facilitating cell death comprising:

administering quantum dots (QDs) conjugated to 5-Aminolevulinic acid (5-ALA) or esters thereof to undesired cells;

co-administering free 5-ALA or esters thereof to the undesired cells; and

physically exciting the QDs conjugated to 5-ALA or esters thereof to induce PpIX fluorescence and generation of reactive oxygen species (ROS) that facilitate cell death.

In an embodiment, there is provided the use of quantum dots (QDs) conjugated to 5-Aminolevulinic acid (5-ALA), for use in the manufacture of a medicament for facilitating cell death comprising:

administering quantum dots (QDs) conjugated to 5-Aminolevulinic acid (5-ALA) or esters thereof to undesired cells;

co-administering free 5-ALA or esters thereof to the undesired cells; and

physically exciting the QDs conjugated to 5-ALA or esters thereof to induce PpIX fluorescence and generation of reactive oxygen species (ROS) that facilitate cell death.

### BRIEF DESCRIPTION OF THE DRAWINGS

FIG. 1 is a figurative illustration of conjugating a QD with 5-ALA.

FIG. 2A-FIG. 2F show the intracellular uptake in SKBR3 human breast cancer cells of passive QDs (FIG. 2A), cRGD-QDs (FIG. 2B), free 5-ALA (FIG. 2C), 5-ALA-QDs (FIG. 2D), 5-ALA-QDs+added free 5-ALA (FIG. 2E), and cRGD-QD+added free 5-ALA (FIG. 2F) at an incubation time of 5 hours at 5× magnification. The intracellular uptake of passive QDs (FIG. 2G), cRGD-QDs (FIG. 2H), free 5-ALA (FIG. 2I), 5-ALA-QDs (FIG. 2J), 5-ALA-QDs+added free 5-ALA (FIG. 2K), and cRGD-QD+added free 5-ALA (FIG. 2L) at an incubation time of 5 hours is also shown at 100× magnification.

FIG. 3A-FIG. 3E show the intracellular uptake in SKBR3 human breast cancer cells of passive QDs (FIG. 3A), cRGD-QDs (FIG. 3B), free 5-ALA (FIG. 3C), 5-ALA-QDs (FIG. 3D), and 5-ALA-QDs+added free 5-ALA (FIG. 3E) at an incubation time of 16 hours at 5× magnification. The intracellular uptake of passive QDs (FIG. 3F), cRGD-QDs (FIG. 3G), free 5-ALA (FIG. 3H), 5-ALA-QDs (FIG. 3I), and 5-ALA-QDs+added free 5-ALA (FIG. 3J) at an incubation time of 16 hours is also shown at 100× magnification.

FIG. 4A-FIG. 4F show the intracellular uptake in SKBR3 human breast cancer cells of passive QDs (FIG. 4A), Herceptin-QDs+free 5-ALA (FIG. 4B), free 5-ALA (FIG. 4C), 5-ALA-QDs (FIG. 4D), 5-ALA-QDs+added free 5-ALA (FIG. 4E), and passive QDs+added free 5-ALA (FIG. 4F) at an incubation time of 3 hours at 5× magnification. The intracellular uptake in SKBR3 human breast cancer cells of passive QDs (FIG. 4G), Herceptin-QDs+free 5-ALA (FIG. 4H), free 5-ALA (FIG. 4I), 5-ALA-QDs (FIG. 4J), 5-ALA-QDs+added free 5-ALA (FIG. 4K), and passive QDs+added free 5-ALA (FIG. 4L) at an incubation time of 3 hours also shown at 100× magnification.

FIG. 5 is an illustration of one proposed mechanism of signal enhancement.

FIG. 6A and FIG. 6B show the resistance to photo-bleaching of 5-ALA-QD+free 5-ALA (FIG. 6A) versus free 5-ALA (FIG. 6B) after uptake by SKBR3 human breast cancer cells and after 1 minute of irradiation.

FIG. 7A-FIG. 7D compare detection of melanoma human A375 cancer cells after 5 hrs treatment with plain QDs (FIG. 7A), 5-ALA-QDs (50 μg/mL) (FIG. 7B), 5-ALA-QDs (50 μg/mL)+free 5-ALA (0.5 mM) (FIG. 7C), and 5-ALA alone (0.5 mM) (FIG. 7D).

FIG. 8A-FIG. 8C compare detection of human squamous cell carcinoma A431 after 5 hrs treatment with plain QDs (FIG. 8A), 5-ALA alone (0.5 mM) (FIG. 8B), and 5-ALA-QDs (50 μg/mL)+free 5-ALA (0.5 mM) (FIG. 8C).

FIG. 9A-FIG. 9C compare confocal microscopic detection of human brain cancer cells GIN3 glioblastoma after 5 hrs treatment with plain QD (FIG. 9A), 5-ALA-QDs ((100 μg/mL)+5-ALA (1 mM) (FIG. 9B), and free 5-ALA (1 mM) (FIG. 9C).

FIG. 10 shows the results of flow cytometry of human brain cancer cells GIN3 glioblastoma after 5 hrs treatment with plain QD (100 μg/mL), 5-ALA-QDs (100 μg/mL)+5-ALA (1 mM), and free 5-ALA (1 mM) in comparison with control untreated and unlabeled cells.

FIG. 11A-FIG. 11D show the lack of labeling of normal human fibroblast cells (HFF) after 16 hrs of treatment with plain QD (50 μg/mL) (FIG. 11A), 5-ALA-QDs (50 μg/mL) (FIG. 11B), 5-ALA-QDs (50 μg/mL)+free 5-ALA (0.2 mM) (FIG. 11C), and free 5-ALA alone (0.2 mM) (FIG. 11D).

FIG. 12A-FIG. 12H show labelling of 3D cultured human pancreatic Mia-Paca-2 cancer (spheroids) after 16 hrs treatment with plain QD (50 μg/mL) (FIG. 12A and FIG. 12E), 5-ALA-QDs (50 μg/mL) (FIG. 12B and FIG. 12F), 5-ALA-QDs (50 μg/mL)+free 5-ALA (0.2 mM) (FIG. 12C and FIG. 12G), and free 5-ALA alone (0.2 mM) (FIG. 12D and FIG. 12H). ImageJ 1.51w software was used to generate surface plots of the red channel intensity from each image as shown in the lower panels FIG. 12E-FIG. 12H.

FIG. 13A-FIG. 13D show the similar performance of QD conjugates to derivatives of 5-ALA (i.e. a hexyl ester of 5 amino levulenic acid (HALA) by treating cultured human pancreatic Mia-Paca-2 cancer for 5 hrs with plain QD (FIG. 13A), HALA-QDs conjugate (50 μg/mL)(FIG. 13B), HALA-QDs conjugate (50 μg/mL)+free HALA (0.5 mM) (FIG. 13C), or free HALA alone (0.5 mM) (FIG. 13D).

FIG. 14A-FIG. 14D show in vivo imaging of Mia Paca-2 human pancreatic tumors grown on the flank of immunosuppressed nude mice and then treated by intra-tumoral injection of 250 mg/Kg of 5-ALA (FIG. 14A and FIG. 14C) or 20 mg/Kg 5-ALA conjugated QDs dimensioned for emission at 630 nm) (FIG. 14B and FIG. 14D). Imaging was performed on live animals after 5 h post injection using a whole animal imager (FIG. 14A and FIG. 14B) or a hand-held blue flash light and a digital camera (FIG. 14C and FIG. 14D).

### DETAILED DESCRIPTION OF THE INVENTION

Appreciating the shortcomings of existing photodynamic therapy (PDT) utilizing a photosensitizer (PS) along with light to visualize and kill unwanted tissue and cancer cells, the present inventors undertook to enhance the activities of 5-Aminolevulinic acid (5-ALA) and 5-ALA derivatives such as 5-ALA esters conjugated to Quantum Dots (QDs). Provided herein are certain embodiments that provide QD conjugates that feature high safety and biocompatibility profiles. Disclosed herein are QDs conjugated with 5-Aminolevulinic acid (5-ALA) and derivatives thereof such as HALA, and methods for enhancing 5-Aminolevulinic acid (5-ALA) based medical imaging and phototherapy with the use of QDs. In certain embodiments, a biocompatible, non-toxic, fluorescent QDs is conjugated with 5-ALA. In other embodiments, the 5-ALA-QD conjugates are formed of semiconductor materials that are themselves toxic and contribute to the cytotoxicity of the system.

As disclosed in co-pending U.S. patent application Ser. No. 15/235,362, QDs are modified with targeting ligands and are further conjugated to externally linked 5-Aminolevulinic acid (5-ALA), 5-ALA derivatives, or 5-ALA analogs. Subsequent work by the inventor of the co-pending U.S. patent application Ser. No. 15/235,362 surprisingly found that QDs conjugated with 5-ALA enhance the cellular uptake of 5-ALA via the 5-ALA-specific membrane transporters, such as, for example, BETA transporters (GAT-1 to GAT-3, BGT-1 and TAUT), GABA transporters, PEPT1 and PEPT2. As disclosed herein, it has now also been very surprisingly found that emission signals at 630 nm are significantly enhanced when QDs conjugated to 5-ALA are administered together with additional free 5-ALA (non-endogenous and unconjugated 5-ALA).

### ABBREVIATIONS

To facilitate the understanding of this invention, and for the avoidance of doubt in construing the claims herein, a number of terms are defined below. Terms defined herein have meanings as commonly understood by a person of ordinary skill in the areas relevant to the present invention. The terminology used to describe specific embodiments of the invention does not delimit the invention, except as outlined in the claims.

DCC dicyclohexylcarbodiimide

DCM dichloromethane

DIC diisopropylcarbodiimide

EDC 1-ethyl-3-(-3-dimethylaminopropyl) carbodiimide hydrochloride

HMMM hexamethoxymethylmelamine

In(MA)₃ indium myristate

QD Quantum Dot

sulfo-NHS sulfo derivative of N-hydroxysuccinimide

SMCC succinimidyl 4-(N-maleimidomethyl) cyclohexane-1-carboxylate

(TMS)₃P tris(trimethylsilyl) phosphine

5-ALA 5-Aminolevulinic acid

The terms such as “a,” “an,” and “the” are not intended to refer to a singular entity unless explicitly so defined, but include the general class of which a specific example may be used for illustration. The use of the terms “a” or “an” when used in conjunction with “comprising” in the claims and/or the specification may mean “one” but may also be consistent with “one or more,” “at least one,” and/or “one or more than one.”

The use of the term “or” in the claims is used to mean “and/or” unless explicitly indicated to refer to alternatives as mutually exclusive. Thus, unless otherwise stated, the term “or” in a group of alternatives means “any one or combination of” the members of the group. Further, unless explicitly indicated to refer to alternatives as mutually exclusive, the phrase “A, B, and/or C” means embodiments having element A alone, element B alone, element C alone, or any combination of A, B, and C taken together.

Similarly, for the avoidance of doubt and unless otherwise explicitly indicated to refer to alternatives as mutually exclusive, the phrase “at least one of” when combined with a list of items, means a single item from the list or any combination of items in the list. For example, and unless otherwise defined, the phrase “at least one of A, B and C,” means “at least one from the group A, B, C, or any combination of A, B and C.” Thus, unless otherwise defined, the phrase requires one or more, and not necessarily not all, of the listed items.

The terms “comprising” (and any form thereof such as “comprise” and “comprises”), “having” (and any form thereof such as “have” and “has”), “including” (and any form thereof such as “includes” and “include”) or “containing” (and any form thereof such as “contains” and “contain”) are inclusive or open-ended and do not exclude additional, unrecited elements or method steps.

The term “effective” as used in the specification and claims, means adequate to provide or accomplish a desired, expected, or intended result.

The terms “about” or “approximately” are defined as being close to as understood by one of ordinary skill in the art, and in one non-limiting embodiment the terms are defined to be within 10%, within, 5%, within 1%, and in certain aspects within 0.5%.

Forms of administration may include preparations for parenteral administration by subcutaneous, intradermal, intramuscular or intravenous administration (e.g., injectable administration) such as sterile suspensions or emulsions. Gastrointestinal routes of administration may also be employed such as for gastrointestinal cancerous and precancerous conditions such as for example Barrett's Esophagus. Compositions may be topically administered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired. Standard pharmaceutical texts, such as “Remington's Pharmaceutical Sciences,” 1990 may be consulted to prepare suitable preparations, without undue experimentation.

As used herein, the terms “pharmaceutically acceptable carrier” and “pharmaceutically acceptable vehicle” are interchangeable and refer to a fluid vehicle for containing 5-ALA-QD or 5-ALA ester-QD that can be injected into a host without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers sugars and amino acids, preservatives, wetting agents, emulsifying agents, pH buffering agents, viscosity enhancing additives, colors and the like.

The terms “co-administered”, “a combination of” and “in combination with” include administration with 5-ALA-QD or 5-ALA ester-QD with free exogenously administered free 5-ALA or free 5-ALA ester either simultaneously, concurrently or sequentially in any order without specific time limits so long as the “co-administered” agents are present in measurable amounts in a single patient at a given time. In certain embodiments, the therapeutic agents are in the same composition or unit dosage form while in other embodiments, the therapeutic agents are in separate compositions or unit dosage forms. The terms “active agent,” “drug,” “therapeutic agent,” and synonymous terms according to those of skill in the art are used interchangeably herein.

QDs are fluorescent semiconductor nanoparticles with unique optical properties. QD represent a particular very small size form of semiconductor material in which the size and shape of the particle results in quantum mechanical effects upon light excitation. Generally, larger QDs such as having a radius of 5-6 nm will emit longer wavelengths in orange or red emission colors and smaller QDs such as having a radius of 2-3 nm emit shorter wavelengths in blue and green colors, although the specific colors and sizes depend on the composition of the QD. QDs shine around 20 times brighter and are many times more photo-stable than any of the conventional fluorescent dyes (like indocyanine green (ICG)). Importantly, QD residence times are longer due to their chemical nature and nano-size. QDs can absorb and emit much stronger light intensities. In certain embodiments, the QD can be equipped with more than one binding tag, forming bi- or tri-specific nano-devices. The unique properties of QDs enable several medical applications that serve unmet needs.

In embodiments presented herein, the QDs are functionalized to present a hydrophilic outer layer or corona that permits use of the QDs in the aqueous environment, such as, for example, in vivo and ex vivo applications in living cells. Such QDs are termed water soluble QDs.

For one example of delivery of 5-ALA QD conjugates systemically in the treatment of tumors, the 5-ALA QD conjugate is administered parenterally and allowed to circulate until the drug loaded QD has concentrated in the tumor. Particulates such as QDs are expected to accumulate in the vasculature of tumors after repeated passes through the circulation because the spongey vasculature of tumors is known to trap particulates in circulation to levels higher than those existing systemically. This phenomenon is known as Enhanced Permeability and Retention effect (EPR). In one embodiment the QD includes polyethylene glycol (PEG) moieties that reduce removal of the QD by the reticuloendothelial system as they circulate such that the QD is allowed to accumulate in the tumor. Once the QDs are delivered to the tumor, the loaded drug is released by administering light into the local environment of the target tissue either by open or closed procedures. In one embodiment of light administered into the local environment of the tumor, the tumor is an intra-abdominal tumor and the light source is introduced into the abdomen endoscopically. In other embodiments, the 5-ALA QD conjugate is injected directly into the tumor tissue and drug is released by administering light into the local environment of the target tumor either by open or closed procedures.

In one embodiment the QDs may be surface equipped with a conjugation capable function (for example, COOH, OH, NH₂, SH, azide, alkyne). In one exemplified embodiment, the water soluble non-toxic QD is or becomes carboxyl functionalized. For example, the COOH-QD may be linked to the amine terminus of 5-ALA using a carbodiimide linking technology employing water-soluble 1-ethyl-3-(-3-dimethylaminopropyl) carbodiimide hydrochloride (EDC). The carboxyl functionalized QD is mixed with EDC to form an active O-acylisourea intermediate that is then displaced by nucleophilic attack from primary amino groups on the 5-ALA in the reaction mixture. If desired, a sulfo derivative of N-hydroxysuccinimide (sulfo-NHS) is added during the reaction with the primary amine bearing molecule. With the sulfo-NHS addition, the EDC couples NHS to carboxyls, forming an NHS ester that is more stable than the O-acylisourea intermediate while allowing for efficient conjugation to primary amines at physiologic pH. In either event, the result is a covalent bond between the QD and the amine bearing molecule. Other chemistries like Suzuki-Miyaura cross-coupling, (succinimidyl 4-(N-maleimidomethyl) cyclohexane-1-carboxylate) (SMCC), or aldehyde based reactions may alternatively be used.

Methods of synthesizing core and core-shell nanoparticles are disclosed, for example, in co-owned U.S. Pat. Nos. 7,867,556, 7,867,557, 7,803,423, 7,588,828, and 6,379,635. The contents of each of the forgoing patents are hereby incorporated by reference, in their entirety. U.S. Pat. Nos. 9,115,097, 8,062,703, 7,985,446, 7,803,423, and 7,588,828, and U.S. Publication Nos. 2010/0283005, 2014/0264196, 2014/0277297 and 2014/0370690, the entire contents of each of which are hereby incorporated by reference, describe methods of producing large volumes of high quality monodisperse QDs.

In certain embodiments, the light responsive 5-ALA QD conjugate utilizes a water soluble QD nanoparticle that is considered a “core only” nanoparticle formed of a semiconductor material but lacking an inorganic shell of a different semiconductor material. Core only QDs are capable of light absorption but in some cases do not exert strong fluorescence emission and thus have been disfavored for purposes where light emission is the purpose of the QD. When used for 5-ALA delivery, core only QDs that lack strong fluorescence emission but have sufficient energy absorption for structural perturbation and 5-ALA release upon light excitation may be utilized.

In other embodiments, a core/shell particle is utilized having a central region or “core” of at least one semiconductor composition buried in or coated by one or more outer layers or “shell” of distinctly different semiconductor compositions. As an example, the core may be comprised of an alloy of In, P, Zn and S involving molecular seeding of indium-based QDs over a ZnS molecular cluster followed by formation of a shell of ZnS.

In still other embodiments, the water soluble QD nanoparticle employed comprises an alloyed semiconductor material having a bandgap value or energy (E_{g}) that increases outwardly by graded alloying in lieu of production of a core/shell QD. The band gap energy (E_{g}), is the minimum energy required to excite an electron from the ground state valence energy band into the vacant conduction energy band.

The graded alloy QD composition is considered “graded” in elemental composition from at or near the center of the particle to the outermost surface of the QD rather than formed as a discrete core overlaid by a discrete shell layer. An example would be an In₁₋ₓP_{1-y}ZₙₓS_{y}, graded alloy QD wherein the x and y increase gradually from 0 to 1 from the center of the QD to the surface. In such example, the band gap of the QD would gradually change from that of pure InP towards the center to that of a larger band gap value of pure ZnS at the surface. Although the band gap of a nanoparticle is dependent on particle size, the bulk band gap of ZnS is wider than that of InP such that the band gap of the graded alloy would gradually increase from an inner aspect of the QD to the surface.

A one-pot synthesis process may be employed as a modification of the molecular seeding process described in Example 1 herein. This may be achieved by gradually decreasing the amounts of indium myristate (In(MA)₃) and tris (trimethylsilyl) phosphine (TMS)₃P added to the reaction solution to maintain particle growth, while adding increasing amounts of zinc and sulfur precursors during a process such as is described for generation of the “core” particle of Example 1. Thus, in one example a dibutyl ester and a saturated fatty acid are placed into a reaction flask and degassed with heating. Nitrogen is introduced and the temperature is increased. A molecular cluster, such as for example a ZnS molecular cluster [Et₃NH]₄ [Zn₁₀S₄(SPh)₁₆], is added with stirring. The temperature is increased as graded alloy precursor solutions are added according to a ramping protocol that involves addition of gradually decreasing concentrations of a first semiconductor material and gradually increasing concentrations of a second semiconductor material. For example, the ramping protocol may begin with additions of In(MA)₃ and (TMS)₃P dissolved in a dicarboxylic acid ester (such as for example di-n-butylsebacate ester) wherein the amounts of added In(MA)₃ and (TMS)₃P gradually decrease over time to be replaced with gradually increasing concentration of sulfur and zinc compounds such as (TMS)₂S and zinc acetate. As the added amounts of In(MA)₃ and (TMS)₃P decrease, gradually increasing amounts of (TMS)₂S dissolved in a saturated fatty acid (such as for example myristic or oleic acid) and a dicarboxylic acid ester (such as di-n-butyl sebacate ester) are added together with the zinc acetate. The following reactions will result in the increasing generation of ZnS compounds. As the additions continue, QD particles of a desired size with an emission maximum gradually increasing in wavelength are formed wherein the concentrations of InP and ZnS are graded with the highest concentrations of InP towards a center of the QD particle and the highest concentrations of ZnS on an outer layer of the QD particle. Further additions to the reaction are stopped when the desired emission maximum is obtained and the resultant graded alloy particles are left to anneal followed by isolation of the particles by precipitation and washing.

A nanoparticle's compatibility with a medium as well as the nanoparticle's susceptibility to agglomeration, photo-oxidation and/or quenching, is mediated largely by the surface composition of the nanoparticle. The coordination about the final inorganic surface atoms in any core, core-shell or core-multi shell nanoparticle may be incomplete, with highly reactive “dangling bonds” on the surface, which can lead to particle agglomeration. This problem is overcome by passivating (capping) the “bare” surface atoms with protecting organic groups, referred to herein as capping ligands or a capping agent. The capping or passivating of particles prevents particle agglomeration from occurring but also protects the particle from its surrounding chemical environment and provides electronic stabilization (passivation) to the particles, in the case of core material. The capping ligands may be but are not limited to a Lewis base bound to surface metal atoms of the outermost inorganic layer of the particle. The nature of the capping ligand largely determines the compatibility of the nanoparticle with a particular medium. Capping ligand may be selected depending on desired characteristics. Types of capping ligands that may be employed include, but are not restricted to, thiol groups, carboxyl, amine, phosphine, phosphine oxide, phosphonic acid, phosphinic acid, imidazole, OH, thio ether, and calixarene groups. With the exception of calixarenes, all of these capping ligands have head groups that can form anchoring centers for the capping ligands on the surface of the particle. The body of the capping ligand can be a linear chain, cyclic, or aromatic. The capping ligand itself can be large, small, oligomeric or polydentate. The nature of the body of the ligand and the protruding side that is not bound onto the particle, together determine if the ligand is hydrophilic, hydrophobic, amphiphilic, negative, positive or zwitterionic.

In many QD materials, the capping ligands are hydrophobic (for example, alkyl thiols, fatty acids, alkyl phosphines, alkyl phosphine oxides, and the like). Thus, the nanoparticles are typically dispersed in hydrophobic solvents, such as toluene, following synthesis and isolation of the nanoparticles. Such capped nanoparticles are typically not dispersible in more polar media. If surface modification of the QD is desired, the most widely used procedure is known as ligand exchange. Lipophilic ligand molecules that coordinate to the surface of the nanoparticle during core synthesis and/or shelling procedures may subsequently be exchanged with a polar/charged ligand compound. An alternative surface modification strategy intercalates polar/charged molecules or polymer molecules with the ligand molecules that are already coordinated to the surface of the nanoparticle. However, while certain ligand exchange and intercalation procedures render the nanoparticle more compatible with aqueous media, they may result in materials of lower quantum yield (QY) and/or substantially larger size than the corresponding unmodified nanoparticle.

For in vivo and ex vivo purposes, QDs with low toxicity profiles are desirable if not required. Thus, for some purposes, the QD is “non-toxic” as defined as substantially free of toxic heavy metals such as cadmium, lead and arsenic (e.g., contains less than 5 wt. %, such as less than 4 wt. %, less than 3 wt. %, less than 2 wt. %, less than 1 wt. %, less than 0.5 wt. %, less than 0.1 wt. %, less than 0.05 wt. %, or less than 0.01 wt. % of heavy metals such as cadmium, lead and arsenic) or is free of heavy metals such as cadmium, lead and arsenic. In one embodiment, reduced toxicity QDs that lack heavy metals such as cadmium, lead and arsenic are provided.

The unique properties of QDs enable several potential medical applications including unmet in vivo and ex vivo diagnostics in living cells. One of the major concerns regarding the medical applications of QDs has been that the majority of research has focused on QDs containing toxic heavy metals such as cadmium, lead or arsenic. The biologically compatible and water-soluble heavy metal-free QDs described in certain embodiments herein can safely be used in medical applications both in vivo and ex vivo. In certain embodiments, in vivo compatible water dispersible cadmium-free QDs are provided that have a hydrodynamic size of 10-20 nm (for comparison, within the range of the dimensional size of a full IgG2 antibody). In one embodiment, the in vivo compatible water dispersible cadmium-free QDs are produced. In certain embodiments, the in vivo compatible water dispersible cadmium-free QDs are carboxyl functionalized and further derivatized with a ligand or a ligand binding moiety.

In certain aspects the ligands are selected from one or more of the group consisting of antibodies, streptavidin, nucleic acids, lipids, saccharides, drug molecules, proteins, peptides, and amino acids. In certain aspects the detecting is used for imaging and detecting one or more of angiogenesis, tumor demarcation, tumor metastasis, diagnostics in vivo, and lymph node progression while in other aspects the detecting is used in one or more of immunochemistry, immunofluorescence, DNA sequence analysis, fluorescence resonance energy transfer, flow cytometry, fluorescence activated cell sorting, and high-throughput screening. In certain aspects at least one of the ligands has specificity for a target selected from the group consisting of EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand. In particular aspects a multi-ligand nano-device is provided having at least a 5-ALA and at least one target specific ligand.

In particular embodiment the targeting ligand is an antibody. As used herein the term “antibody” includes both intact immunoglobulin molecules as well as portions, fragments, and derivatives thereof, such as, for example, Fab, Fab′, F(ab)₂, Fv, Fsc, CDR regions, or any portion of an antibody that is capable of binding an antigen or epitope including chimeric antibodies that are bi-specific or that combine an antigen binding domain originating with an antibody with another type of polypeptide. The term antibody thus includes monoclonal antibodies (mAb), chimeric antibodies, humanized antibodies, as well as fragments, portions, regions, or derivatives thereof, provided by any known technique including but not limited to, enzymatic cleavage and recombinant techniques. The term “antibody” as used herein also includes single-domain antibodies (sdAb) and fragments thereof that have a single monomeric variable antibody domain (V_{H}) of a heavy-chain antibody. sdAb, which lack variable light (V_{L}) and constant light (CO chain domains are natively found in camelids (V_{H}H) and cartilaginous fish (V_{NAR}) and are sometimes referred to as “Nanobodies” by the pharmaceutical company Ablynx who originally developed specific antigen binding sdAb in llamas. The modifier “monoclonal” indicates the character of the antibody as being obtained from a substantially homogeneous population of antibodies, and is not to be construed as requiring production of the antibody by any particular method.

In vitro and in vivo toxicology studies with the in vivo compatible water dispersible cadmium-free or non-toxic QDs disclosed herein showed them to be are at least 20 times less cytotoxic than commercially available cadmium-based QDs, and no toxicity signs were observed on animal models at multiple times higher than useful doses. Furthermore, the in vivo compatible water dispersible cadmium-free QD nanoparticles herein provided showed no hemolytic effect and no complement C3 activation, indicating a favorable clinical compatibility profile.

Examples of cadmium, lead and arsenic free nanoparticles include nanoparticles comprising semiconductor materials, e.g., ZnS, ZnSe, ZnTe, InP, InSb, AlP, AlS, AlSb, GaN, GaP, GaSb, AgInS₂, CuInS₂, Si, Ge, and alloys and doped derivatives thereof, particularly, nanoparticles comprising cores of one of these materials and one or more shells of another of these materials.

It is noted that nanoparticles that include a single semiconductor material, e.g., CdS, CdSe, ZnS, ZnSe, InP, GaN, etc. may have relatively low quantum efficiencies because of non-radiative electron-hole recombination that occurs at defects and dangling bonds at the surface of the nanoparticles. In order to at least partially address these issues, the nanoparticle cores may be at least partially coated with one or more layers (also referred to herein as “shells”) of a material different than that of the core, for example a different semiconductor material than that of the “core.” The material included in the one or more shells may incorporate ions from any one or more of groups 2 to 16 of the periodic table. When a nanoparticle has two or more shells, each shell may be formed of a different material. In an exemplary core/shell material, the core is formed from one of the materials specified above and the shell includes a semiconductor material of larger band-gap energy and similar lattice dimensions as the core material. Exemplary shell materials include, but are not limited to, ZnS, ZnO, ZnSe, MgS, MgSe, MgTe and GaN. One example of a multi-shell nanoparticle is InP/ZnS/ZnO. The confinement of charge carriers within the core and away from surface states provides nanoparticles of greater stability and higher quantum yield.

However, while it may be desirable to have QDs that lack toxic heavy metals, it has proved particularly difficult to modify the surface of cadmium-free nanoparticles. Cadmium-free nanoparticles readily degrade when methods such as the aforementioned ligand exchange methods are used to modify the surface of such cadmium-free nanoparticles. For example, attempts to modify the surface of cadmium-free nanoparticles have been observed to cause a significant decrease in the luminescence QY of such nanoparticles. For certain in vivo purposes disclosed herein, surface-modified cadmium-free nanoparticles with high QY are required. The high QY cadmium-free water dispersible nanoparticles disclosed herein have a QY greater than about 20%, greater than about 25%, greater than about 30%, greater than about 35%, or greater than about 40%. For certain in vivo embodiments, heavy metal-free semiconductor indium-based nanoparticles or nanoparticles containing indium and/or phosphorus are preferred.

In certain embodiments, non-toxic QD nanoparticles are surface modified to enable them to be water soluble and to have surface moieties that allow derivatization by exposing them to a ligand interactive agent to effect the association of the ligand interactive agent and the surface of the QD. The ligand interactive agent can comprise a chain portion and a functional group having a specific affinity for, or reactivity with, a linking/crosslinking agent, as described below. The chain portion may be, for example, an alkane chain. Examples of functional groups include nucleophiles such as thio groups, hydroxyl groups, carboxamide groups, ester groups, and a carboxyl groups. The ligand interactive agent may, or may not, also comprise a moiety having an affinity for the surface of a QD. Examples of such moieties include thiols, amines, carboxylic groups, and phosphines. If the ligand interactive group does not comprise such a moiety, the ligand interactive group can associate with the surface of the nanoparticle by intercalating with capping ligands. Examples of ligand interactive agents include C₈₋₂₀ fatty acids and esters thereof, such as for example isopropyl myristate.

It should be noted that the ligand interactive agent may be associated with a QD nanoparticle simply as a result of the processes used for the synthesis of the nanoparticle, obviating the need to expose nanoparticle to additional amounts of ligand interactive agents. In such case, there may be no need to associate further ligand interactive agents with the nanoparticle. Alternatively, or in addition, QD nanoparticle may be exposed to ligand interactive agent after the nanoparticle is synthesized and isolated. For example, the nanoparticle may be incubated in a solution containing the ligand interactive agent for a period of time. Such incubation, or a portion of the incubation period, may be at an elevated temperature to facilitate association of the ligand interactive agent with the surface of the nanoparticle. Following association of the ligand interactive agent with the surface of nanoparticle, the QD nanoparticle is exposed to a linking/crosslinking agent and a surface modifying ligand. The linking/crosslinking agent includes functional groups having specific affinity for groups of the ligand interactive agent and with the surface modifying ligand. The ligand interactive agent-nanoparticle association complex can be exposed to a linking/crosslinking agent and surface modifying ligand sequentially. For example, the nanoparticle might be exposed to the linking/crosslinking agent for a period of time to effect crosslinking, and then subsequently exposed to the surface modifying ligand to incorporate it into the ligand shell of the nanoparticle. Alternatively, the nanoparticle may be exposed to a mixture of the linking/crosslinking agent and the surface-modifying ligand thus effecting crosslinking and incorporating surface modifying ligand in a single step.

In one embodiment, QD precursors are provided in the presence of a molecular cluster compound under conditions whereby the integrity of the molecular cluster is maintained and acts as a well-defined prefabricated seed or template to provide nucleation centers that react with the chemical precursors to produce high quality nanoparticles on a sufficiently large scale for industrial application.

Suitable types of QDs useful in the present invention include, but are not limited to, core materials comprising the following types (including any combination or alloys or doped derivatives thereof):

IIA-VIB (2-16) material, incorporating a first element from group 2 of the periodic table and a second element from group 16 of the periodic table and also including ternary and quaternary materials and doped materials. Suitable nanoparticle materials include, but are not limited to: MgS, MgSe, MgTe, CaS, CaSe, CaTe, SrS, SrSe, SrTe, BaS, BaSe, BaTe.

II-V material incorporating a first element from group 12 of the periodic table and a second element from group 15 of the periodic table and also including ternary and quaternary materials and doped materials. Suitable nanoparticle materials include, but are not limited to: Zn₃P₂, Zn₃As₂, Cd₃P₂, Cd₃As₂, Cd₃N₂, Zn₃N₂.

II-VI material incorporating a first element from group 12 of the periodic table and a second element from group 16 of the periodic table and also including ternary and quaternary materials and doped materials. Suitable nanoparticle materials include, but are not limited to: CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, CdZnSeS, CdZnSeTe, CdHgSeS, CdHgSeTe, CdHgSTe, HgZnSeS, and HgZnSeTe.

III-V material incorporating a first element from group 13 of the periodic table and a second element from group 15 of the periodic table and also including ternary and quaternary materials and doped materials. Suitable nanoparticle materials include, but are not limited to: BP, AlP, AlSb; GaN, GaP, GaSb; InN, InP, InSb, AlN, and BN.

III-IV material incorporating a first element from group 13 of the periodic table and a second element from group 14 of the periodic table and also including ternary and quaternary materials and doped materials. Suitable nanoparticle materials include, but are not limited to: B₄C, Al₄C₃, Ga₄C, Si, SiC.

III-VI material incorporating a first element from group 13 of the periodic table and a second element from group 16 of the periodic table and also including ternary and quaternary materials. Suitable nanoparticle materials include, but are not limited to: Al₂S₃, Al₂Se₃, Al₂Te₃, Ga₂S₃, Ga₂Se₃, GeTe; In₂S₃, In₂Se₃, Ga₂Te₃, In₂Te₃, InTe.

IV-VI material incorporating a first element from group 14 of the periodic table and a second element from group 16 of the periodic table, and also including ternary and quaternary materials and doped materials. Suitable nanoparticle materials include, but are not limited to: PbS, PbSe, PbTe, Sb₂Te₃, SnS, SnSe, SnTe.

Suitable nanoparticle material can incorporate a first element from any group in the transition metal of the periodic table and a second element from group 16 of the periodic table and also including ternary and quaternary materials and doped materials. For example, a material incorporates a first element from group 11 of the periodic table, a second element from group 13 of the periodic table and a third element from group 16 of the periodic table, and including quaternary, higher order and doped materials. Suitable nanoparticle materials include, but are not limited to: CuInS₂, CuInSe₂, CuGaS₂, CuGaSe₂, AgInS₂, AgInSe₂, NiS, CrS and AgS.

In one embodiment, the QDs useful in the present invention include, but are not limited to, core materials comprising AgS.

In one embodiment of any of the QDs described herein, the nanoparticle comprises a II-IV material, a III-V material, a material, or any alloy or doped derivative thereof.

In one embodiment, the nanoparticle material comprises a II-IV material, a III-V material, and any alloy or doped derivative thereof.

In one embodiment of any of the QDs described herein, the nanoparticle comprises a III-V material, or any alloy or doped derivative thereof.

The term doped nanoparticle for the purposes of specifications and claims refers to nanoparticles of the above and a dopant comprising one or more main group or rare earth elements, this most often is a transition metal or rare earth element, such as but not limited to zinc sulfide with manganese, such as ZnS nanoparticles doped with Mn⁺.

In one embodiment, the QDs is substantially free of heavy metals such as cadmium (e.g., contains less than 5 wt. %, such as less than 4 wt. %, less than 3 wt. %, less than 2 wt. %, less than 1 wt. %, less than 0.5 wt. %, less than 0.1 wt. %, less than 0.05 wt. %, or less than 0.01 wt. % of heavy metals such as cadmium) or is free of heavy metals such as cadmium.

For in vivo applications, heavy metal-free semi-conductor indium-based nanoparticles or nanoparticles containing indium and/or phosphorus are preferred.

In an embodiment, any of the QDs described herein include a first layer including a first semiconductor material provided on the nanoparticle core. A second layer including a second semiconductor material may be provided on the first layer.

### Synthesis

The following synthesis steps may be used for conjugation. Linkers may be used to form an amide group between the carboxyl functions on the nanoparticles and the amine end groups on 5-ALA. Known linkers, such as a thiol anchoring groups directly on the inorganic surface of the QDs can be used. Standard coupling conditions can be employed and will be known to a person of ordinary skill in the art. For example, suitable coupling agents include, but are not limited to, carbodiimides, such as dicyclohexylcarbodiimide (DCC), diisopropylcarbodiimide (DIC), and 1-(3-dimethylaminopropyl)-3-ethylcarbodiimide hydrochloride (EDC). In one embodiment, the coupling agent is EDC.

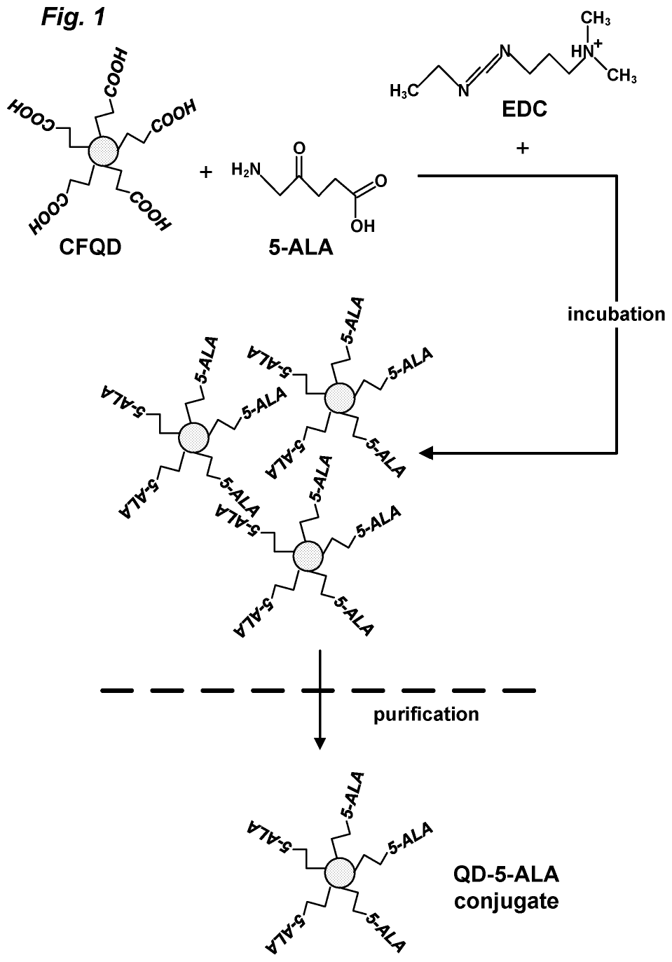

In an example, as illustrated in FIG. 1, the QDs bearing a carboxyl end group and 5-ALA may be mixed in a solvent. A coupling agent, such as EDC, may be added to the mixture. The reaction mixture may be incubated. Standard incubation conditions for coupling can be employed. For example, the coupling conditions may be a solution in the range of 0.5 to 4 hours. The temperature range of the coupling conditions may be in the range of 0° C. to 200° C. The coupling conditions may be constant or varied during the reaction. For example, the reaction conditions may be 130° C. for one hour then raised to 140° C. for three hours.

The crude polymerizable ligand nanoparticle conjugate may be subject to purification and/or isolation. Standard solid state purification methods may be used. Several cycles of filtering and washing with a suitable solvent may be necessary to remove excess unreacted functionalized ligand and/or coupling agents.

In another aspect, one embodiment provides a process for preparing a ligand nanoparticle conjugate for according to any of the embodiments described herein. In one embodiment, the process comprises: i) coupling QDs with 5-ALA or 5-ALA esters to provide QD-5-ALA (or 5-ALA ester) conjugates, wherein the QD comprises a core semiconductor material, and an outer layer, wherein the outer layer comprises a carboxyl group. In one embodiment, coupling step i) comprises (a) reacting a carboxyl group in the outer layer with a carbodiimide linker to activate the carboxyl group, and b) reacting the activated carboxyl group with 5-ALA.

In an additional embodiment, the process further comprises: ii) purifying the QD-5-ALA (or 5-ALA ester) conjugate. In an additional embodiment, the process further comprises: iii) isolating the QD-5-ALA (or 5-ALA ester) conjugate. In one embodiment, the process comprises steps i), ii) and iii).

The following examples are include for the sake of completeness of disclosure and to illustrate the methods of making the compositions and composites of the present invention as well as to present certain characteristics of the compositions. In no way are these examples intended to limit the scope or teaching of this disclosure.

### Example 1

### Synthesis of Non-Toxic Quantum Dots

A molecular seeding process was used to generate non-toxic QDs. Briefly, the preparation of non-functionalized indium-based quantum dots with emission in the range of 500-700 nm was carried out as follows: Dibutyl ester (approximately 100 ml) and myristic acid (MA) (10.06 g) were placed in a three-neck flask and degassed at ˜70° C. under vacuum for 1 h. After this period, nitrogen was introduced and the temperature was increased to ˜90° C. Approximately 4.7 g of a ZnS molecular cluster [Et₃NH]₄[Zn₁₀S₄(SPh)₁₆] was added, and the mixture was stirred for approximately 45 min. The temperature was then increased to ˜100° C., followed by the drop-wise additions of In(MA)₃ (1M, 15 ml) followed by trimethylsilyl phosphine (TMS)₃P (1M, 15 ml). The reaction mixture was stirred while the temperature was increased to ˜140° C. At 140° C., further drop-wise additions of In(MA)₃ dissolved in di-n-butylsebacate ester (1M, 35 ml) (left to stir for 5 min) and (TMS)₃P dissolved in di-n-butylsebacate ester (1M, 35 ml) were made. The temperature was then slowly increased to 180° C., and further dropwise additions of In(MA)₃ (1M, 55 ml) followed by (TMS)₃P (1M, 40 ml) were made. By addition of the precursor in this manner, indium-based particles with an emission maximum gradually increasing from 500 nm to 720 nm were formed. The reaction was stopped when the desired emission maximum was obtained and left to stir at the reaction temperature for half an hour. After this period, the mixture was left to anneal for up to approximately 4 days (at a temperature ˜20-40° C. below that of the reaction). A UV lamp was also used at this stage to aid in annealing.

The particles were isolated by the addition of dried degassed methanol (approximately 200 ml) via cannula techniques. The precipitate was allowed to settle and then methanol was removed via cannula with the aid of a filter stick. Dried degassed chloroform (approximately 10 ml) was added to wash the solid. The solid was left to dry under vacuum for 1 day. This procedure resulted in the formation of indium-based nanoparticles on ZnS molecular clusters. In further treatments, the quantum yields of the resulting indium-based nanoparticles were further increased by washing in dilute hydrofluoric acid (HF). The quantum efficiencies of the indium-based core material ranged from approximately 25%-50%. This composition is considered an alloy structure comprising In, P, Zn and S.

Growth of a ZnS shell: A 20 ml portion of the HF-etched indium-based core particles was dried in a three-neck flask. 1.3 g of myristic acid and 20 ml di-n-butyl sebacate ester were added and degassed for 30 min. The solution was heated to 200° C., and 2 ml of 1 M (TMS)₂S was added drop-wise (at a rate of 7.93 ml/h). After this addition was complete, the solution was left to stand for 2 min, and then 1.2 g of anhydrous zinc acetate was added. The solution was kept at 200° C. for 1 hr. and then cooled to room temperature. The resulting particles were isolated by adding 40 ml of anhydrous degassed methanol and centrifuging. The supernatant liquid was discarded, and 30 ml of anhydrous degassed hexane was added to the remaining solid. The solution was allowed to settle for 5 h and then centrifuged again. The supernatant liquid was collected and the remaining solid was discarded. The QYs of the final non-functionalized indium-based nanoparticle material ranged from approximately 60%-90% in organic solvents.

### Example 2

### Water Soluble Surface Modified QDs

Provided herein is one embodiment of a method for generating and using melamine hexamethoxymethylmelamine (HMMM) modified fluorescent nanoparticles as water soluble QD suitable for conjugation with 5-ALA. The unique melamine-based coating presents excellent biocompatibility, low toxicity and very low non-specific binding. These unique features allow a wide range of biomedical applications both in vitro and in vivo.

One example of preparation of a suitable water soluble nanoparticle is provided as follows: 200 mg of cadmium-free QDs with red emission at 608 nm having as a core material an alloy comprising indium and phosphorus with Zn-containing shells as described in Example 1 was dispersed in toluene (1 ml) with isopropyl myristate (100 microliters). The isopropyl myristate is included as the ligand interactive agent. The mixture was heated at 50° C. for about 1-2 minutes then slowly shaken for 15 hours at room temperature. A toluene solution (4 ml) of HMMM (CYMEL 303, available from Cytec Industries, Inc., West Paterson, N.J.) (400 mg), monomethoxy polyethylene oxide (CH₃O-PEG2000-OH) (400 mg), and salicylic acid (50 mg) was added to the nanoparticle dispersion. The salicylic acid that is included in the functionalization reaction plays three roles, as a catalyst, a crosslinker, and a source for COOH. Due in part to the preference of HMMM for OH groups, many COOH groups provided by the salicylic acid remain available on the QD after crosslinking.

HMMM is a melamine-based linking/crosslinking agent having the following structure:

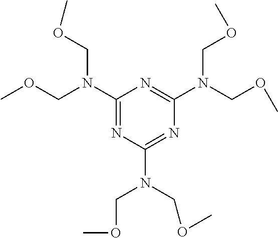

HMMM can react in an acid-catalyzed reaction to crosslink various functional groups, such as amides, carboxyl groups, hydroxyl groups, and thiols.

The mixture was degassed and refluxed at 130° C. for the first hour followed by 140° C. for 3 hours while stirring at 300 rpm with a magnetic stirrer. During the first hour a stream of nitrogen was passed through the flask to ensure the removal of volatile byproducts generated by the reaction of HMMM with nucleophiles. The mixture was allowed to cool to room temperature and stored under inert gas. The surface-modified nanoparticles showed little or no loss in fluorescence quantum yield and no change in the emission peak or full width at half maximum (FWHM) value, compared to unmodified nanoparticles. An aliquot of the surface-modified nanoparticles was dried under vacuum and deionized water was added to the residue. The surface-modified nanoparticles dispersed well in the aqueous media and remained dispersed permanently. In contrast, unmodified nanoparticles could not be suspended in the aqueous medium. The fluorescence QY of the surface-modified nanoparticles according to the above procedure is 40-50%. In typical batches, a quantum yield of 47%±5% is obtained.

In another embodiment, cadmium-free QDs (200 mg) with red emission at 608 nm were dispersed in toluene (1 ml) with cholesterol (71.5 mg). The mixture was heated at 50° C. for about 1-2 minutes then slowly shaken for 15 hours at room temperature. A toluene solution (4 ml) of HMMM (CYMEL 303) (400 mg), monomethoxy polyethylene oxide (CH₃O-PEG2000-OH) (400 mg), guaifenesin (100 mg), dichloromethane (DCM) (2 mL) and salicylic acid (50 mg) was added to the nanoparticle dispersion.

As used herein the compound “guaifenesin” has the following chemical structure:

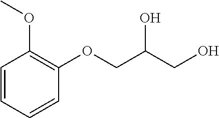

As used herein the compound “salicylic acid” has the following chemical structure:

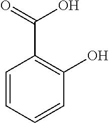

The mixture was degassed and refluxed at 140° C. for 4 hours while stirring at 300 rpm with a magnetic stirrer. As with the prior procedure, during the first hour a stream of nitrogen was passed through the flask to ensure the removal of volatile byproducts generated by the reaction of HMMM with nucleophiles. The mixture was allowed to cool to room temperature and stored under inert gas. An aliquot of the surface-modified nanoparticles was dried under vacuum and deionized water was added to the residue. The pH of the solution was adjusted to 6.5 using a 100 mM KOH solution and the excess non reacted material was removed by three cycles of ultrafiltration using Amicon filters (30 kD). The final aqueous solution was kept refrigerated until use.

It is noteworthy that traditional methods for modifying nanoparticles to increase their water solubility (e.g., ligand exchange with mercapto-functionalized water soluble ligands) are ineffective under mild conditions to render the nanoparticles water soluble. Under harsher conditions, such as heat and sonication, the fraction that becomes water soluble has very low QY (<20%). The instant method, in contrast, provides water soluble nanoparticles with high quantum yield. As defined herein, a high quantum yield is equal to or greater than 40%. In certain embodiments, a high quantum yield is obtained of equal to or greater than 45%. The surface-modified nanoparticles prepared as in this example also disperse well and remain permanently dispersed in other polar solvents, including ethanol, propanol, acetone, methylethylketone, butanol, tripropylmethylmethacrylate, or methylmethacrylate.

### Example 3

### Covalent Conjugation of In Vivo Compatible Water Dispersible Cadmium-Free QDs with an Antibody

In Eppendorf tubes, 1.2 mg carboxyl-functionalised, water-soluble QDs were mixed with 100 μl MES activation buffer (i.e. 25 μl of 50 mg/ml stock into 100 μl MES). The MES buffer is prepared as a 25 mM solution (2-(N-morpholino) ethanesulfonic acid hemisodium salt (MES), Sigma Aldrich) in DI water, pH 4.5. To this was added 33 μl of a fresh EDC solution (30 mg/ml stock in DI water, 1-ethyl-3-(3-dimethylaminopropyl) carbodiimide hydrochloride (EDC), Fisher Scientific) and the solution was mixed. To this was added 4 μl of fresh sulfo-NHS (100 mg/ml stock, ThermoFisher Scientific, in DI water) and the solution was mixed. NanoSep 300K filters (PALL NanoSep 300K Omega ultrafilters) were pre-wetted in 100 μl MES. The MES/EDC/Sulfo-NHS/QD solution was added to the NanoSep 300K filter and topped up 500 μl with MES. The filter was centrifuged at 5000 rpm/15 min. The retained dots were re-dispersed in 50 μl activation buffer and transferred to an Eppendorf tube containing 10 μl of trastuzumab (HERCEPTIN®, 100 mg/ml stock in a 25 mM solution of HEPES buffer, pH 8.5)+40 μl HEPES, pH 8.5. The solution was mixed well and incubated at RT overnight (around 16-18 hours). The solution was quenched with 16 μl of 6-amino caproic acid (6AC) (19.7 mg/100 mM). Note that quenching could be alternatively conducted with other compounds having a primary amine, but 6AC was selected for this embodiment because it has a COOH and can maintain the colloidal stability of the product. The solution was transferred to a pre-whetted Nanosep 300K filter (100 μl 1×PBS) and topped-up to the 500 μl line with 1×PBS. Excess reactants were removed by three cycles of ultrafiltration using Nanosep 300K filters and 1×PBS buffer. Each cycle of centrifugation was 5000 rpm for 20 min with re-dispersal with ˜400 μl of 1×PBS after each cycle. The final concentrated was re-dispersed in 100 μl PBS. Example 4

### Addition of Polymerizable Ligands

In an embodiment, a polymerizable ligand is affixed to the 5-ALA-QD or 5-ALA ester such as a HALA-QD (HALA is a hexyl ester of 5 amino levulenic acid) wherein the polymerizable ligand is polymerized by excitation of the quantum dot nanoparticles with an energy source (e.g., a light source, such as a UV or visible light source).

Suitable polymerizable ligands include, but are not limited to, acrylates, methacrylates, diacetylene, cyanoacrylates, azide/alkyne pairs (click chemistry) and any combination thereof. In one embodiment, the polymerizable ligand is a methacrylate (e.g., 2-aminoethyl methacrylate) or a salt thereof, such as a hydrochloride salt. Suitable acryl based polymerizable ligands include, for example, methacryloyl-L-lysine, 4-methacryloxy-2-hydroxybenzophenone, and salts thereof, and any combination thereof.

In one embodiment, the polymerizable ligand comprises acrylate and methacrylate ligands. For example, quantum dot nanoparticles comprising methacrylate ligands may be polymerized and crosslinked using excitation light to induce exciton formation that can in turn initiate acrylate polymerization.

In additional embodiments, light active monomers (such as, e.g., methacryloyl-L-lysine, 4-methacryloxy-2-hydroxybenzophenone) may be used alone or in combination with one or more standard monomers to enhance the quantum dot nanoparticle's polymerization. In one embodiment, the polymerizable ligand is a cyanoacrylate. In another embodiment, the polymerizable ligand is glycidyl cinnamate, or a derivative thereof. In yet another embodiment, the polymerizable ligand is a diacetylene, e.g., tricosa-10,12-diynoic acid.

Carboxy functionalized QDs are linked to 2-aminoethyl methacrylate hydrochloride using standard EDC chemistry. The resulting dots have pendant methacrylate groups that are delivered to the targeted tissue and polymerized by the excitations of the QDs with an energy source.

In other embodiments carboxy functionalized red QDs were linked to methacryloyl-L-lysine using standard EDC chemistry. The resulting QDs have pendant methacrylate groups that are polymerizable by UV/visible excitation at 300-500 nm. Fluorescence microscopy imaging at 1000× magnification showed that when exposed to 320 nm UV, the nanoparticles aggregated, unlike the ones that were not irradiated. The QDs can be delivered to the targeted tissue and polymerized by the excitations of the QDs with an energy source.

In one example, carboxy functionalized QDs were surface loaded with 4-methacryloxy-2-hydroxybenzophenone (Formula I) using hydrophobic interaction forces as follows. To an amount of 100 mg water soluble dots made in accordance with Examples 1 and 2 were dispersed in 1 mL H₂O, a 1004, solution of 4-methacryloxy-2-hydroxybenzophenone dissolved in DMSO at 100 mg/mL was added with vigorous mixing.

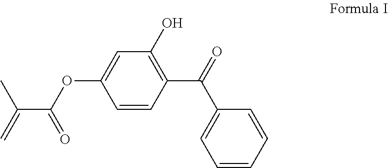 [SMILES: C=C(C)C(=O)Oc1ccc(C(=O)c2ccccc2)c(O)c1]

A clear solution was formed and to which 1 mL of phosphate buffered saline (PBS, pH7.2) was immediately added. The solution remained clear despite the fact that 4-methacryloxy-2-hydroxybenzophenone is insoluble in water. This is an indication that the monomer 4-methacryloxy-2-hydroxybenzophenone was able to form hydrophobic interactions on the surface of the nanoparticles and became dispersed with them. The clear solution was then sterilized using 0.22 μm syringe filter.

A small drop of the polymerizable QD preparation was mounted on a microscope slide, covered with a glass coverslip, and then irradiated for 5 minutes using a 6 Watt handheld UV lamp (UVP, LLC) at 365 nm wavelength. A control slide was prepared in the same manner but was not irradiated. The slides were then examined using a fluorescence microscope. The irradiated sample showed significant aggregation (data not shown).

### Example 5

### Preparation of QD-5-ALA Conjugates

In one example, carboxy functionalized QDs were surface loaded with 5-ALA using hydrophobic interaction forces as follows. To an amount of 100 mg water soluble QD prepared in accordance with Examples 1 and 2 and dimensioned to emit light at 630 nm were dispersed in 1 mL H₂O and a 100 μL solution of 5-ALA dissolved in DMSO at 100 mg/mL was added with vigorous mixing. A solution was formed and to which 1 mL of phosphate buffered saline (PBS, pH7.2) was immediately added.

In another synthesis example, carboxy functionalized QDs were surface loaded with 5-ALA as follows. An amount of 10 mg water soluble dots prepared in accordance with Examples 1 and 2 and dimensioned to emit light at 630 nm was dispersed in 0.5 mL of MES buffer [2-(N-morpholino)ethanesulfonic acid at 25 mM, pH 4.5]. A 100 μL volume of EDC solution in water (1-Ethyl-3-(3-dimethylaminopropyl) carbodiimide (EDC, EDAC or EDCI) 30 mg/mL) was added and followed by a 20 μL solution of Sulfo-NHS (N-hydroxysulfosuccinimide 100 mg/mL in water). The mixture was left at room temperature (RT) for 10 minutes then transferred to an AMICON® 30 kDa centrifugation filter, diluted with 3 mL of MES buffer and spun at 3500 RCF for 30 minutes. The retained nanoparticles in the filter are now activated and were removed and mixed with 100 μL of 5-ALA dissolved in HEPES buffer [HEPES 50 mM, pH7.5] at 50 mg/mL. After overnight incubation at 4° C., the mixture was washed again with two cycles of centrifugation using AMICON® 30 kDa centrifugation filters and PBS, pH7.2. The final aliquot was diluted as needed to form a QD-5-ALA conjugate at 10 mg/mL and kept refrigerated until use.

cRGD-QD conjugates were also prepared. RGD is a peptide with the sequence Arg-Gly-Asp and is known to specifically bind to integrins on the cell membrane, triggering cellular uptake. cRGD is a cyclic version with the sequence cyclo(Arg-Gly-Asp-D-Phe-Lys) (CAS Number: [161552-03-0]) as shown below is known to enable stronger cellular uptake.

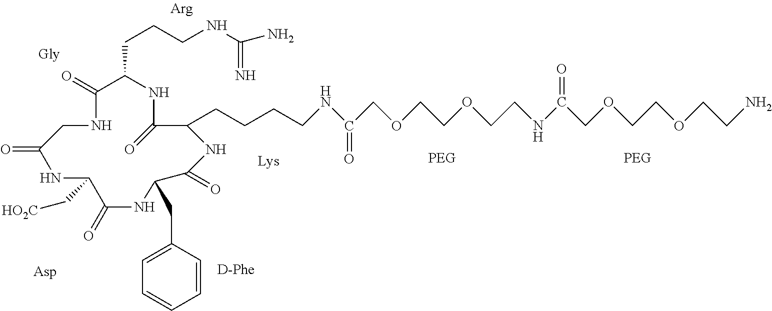 [SMILES: N=C(N)NCCC[C@@H]1NC(=O)C(CCCCNC(=O)COCCOCCNC(=O)COCCOCCN)NC(=O)[C@H](Cc2ccccc2)NC(=O)[C@@H](CC(=O)O)NC(=O)CNC1=O]

A QD conjugate was prepared using cRGD with an amine functionalized linker arm as shown in the structure above [Peptides International, Inc. Louisville, Ky., USA].

The above cRGD was conjugated to the QDs using the immediate above protocol for preparation of 5-ALA conjugates but instead replacing the 5-ALA solution (50 mg/mL solution) with a cRGD solution at 1 mg/mL. The main objective of using a cRGD-QD conjugate was to show that even when the QD uptake was enhanced by cRGD conjugation, the enhancement of 5-ALA signal was not as high as the 5-ALA conjugated QDs, indicating that the enhancement is the resulting from the synthesis of PpIX on or near the QD surface. Hereceptin® (Trastuzumab) QD conjugates were prepared using the similar protocol as described in EXAMPLE 3.

Comparison of 5-ALA Intracellular Uptake:

SKBR3 human breast cancer cells are cultivated in 6 well plates supplied with glass coverslips using McCoy's 5a medium from ATCC (3 mL per well). At about 50% cell growth confluency, QDs or QD conjugates are added to each well to give a final concentration of about ˜50 ug/mL.

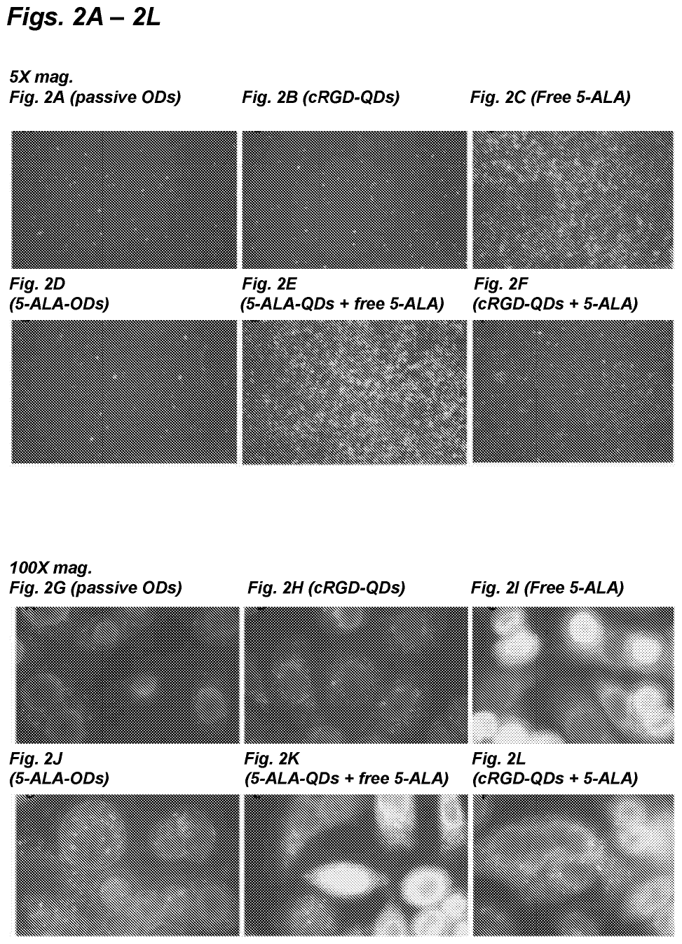

A 5-ALA solution in deionized H₂O at 100 mM was prepared and added when required at a final concentration of 200 uM (6 uL per well). After the predetermined incubation time, cells are fixed using the following protocol. The medium was removed and the cells gently rinsed with PBS buffer (2 mL per well), two mL fixation medium (3.7% formaldehyde in PBS) was added followed by incubation at room temperature (RT) for 15 min. The fixation medium was removed and fresh PBS was added to gently rinse the cells. The coverslips were removed from the wells and mounted on glass slides. Mounting medium (35 uL of glycerol: PBS+0.125 ug/mL Hoechst 33342) was added and the cells were observed with an Olympus BX51 fluorescence microscope using proper filter cubes. Within each study, all microscopic settings (filter, light intensity, exposure time, and magnification) were identical. Microscopic images of the cells after treatment with various types of QDs and/or 5-ALA are shown in FIGS. 2A-2L through FIGS. 4A-4K.

FIG. 2A-FIG. 2F show the intracellular uptake in SKBR3 human breast cancer cells of passive QDs (FIG. 2A), cRGD-QDs (FIG. 2B), free 5-ALA (FIG. 2C), 5-ALA-QDs (FIG. 2D), 5-ALA-QDs+added free 5-ALA (FIG. 2E), and cRGD-QD+added free 5-ALA (FIG. 2F) at an incubation time of 5 hours at 5× magnification. The intracellular uptake of passive QDs (FIG. 2G), cRGD-QDs (FIG. 2H), free 5-ALA (FIG. 2I), 5-ALA-QDs (FIG. 2J), 5-ALA-QDs+added free 5-ALA (FIG. 2K), and cRGD-QD+added free 5-ALA (FIG. 2L) at an incubation time of 5 hours is also shown at 100× magnification.

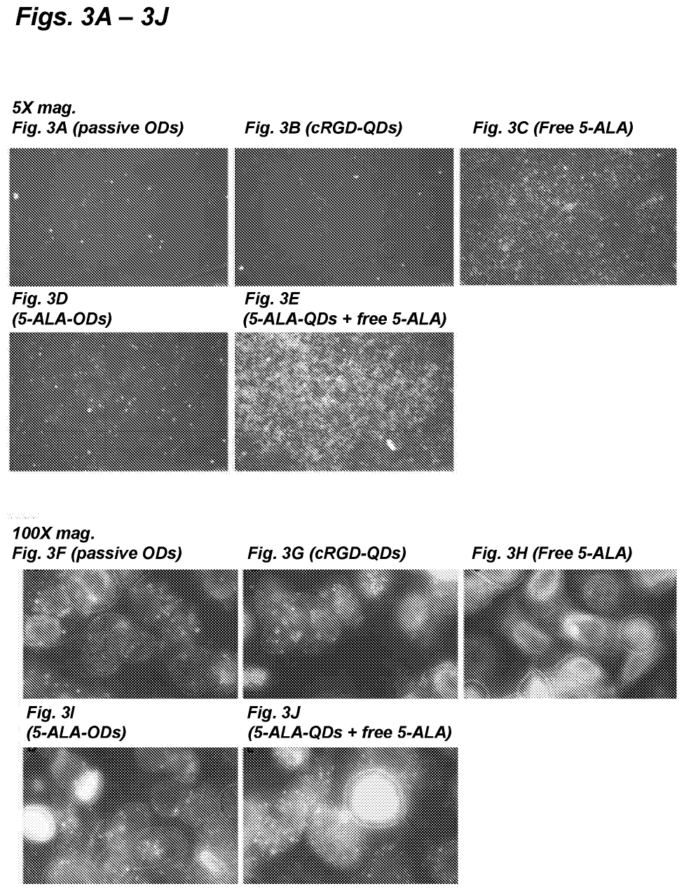

FIG. 3A-FIG. 3E show the intracellular uptake in SKBR3 human breast cancer cells of passive QDs (FIG. 3A), cRGD-QDs (FIG. 3B), free 5-ALA (FIG. 3C), 5-ALA-QDs (FIG. 3D), and 5-ALA-QDs+added free 5-ALA (FIG. 2E) at an incubation time of 16 hours at 5× magnification. The intracellular uptake of passive QDs (FIG. 3F), cRGD-QDs (FIG. 3G), free 5-ALA (FIG. 3H), 5-ALA-QDs (FIG. 3I), and 5-ALA-QDs+added free 5-ALA (FIG. 3J) at an incubation time of 16 hours is also shown at 100× magnification.

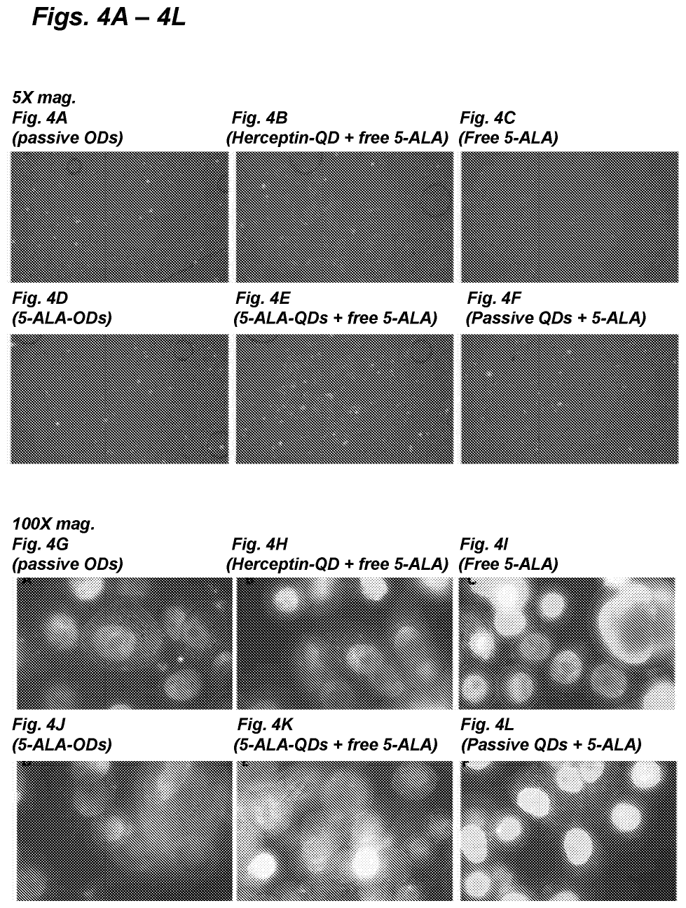

FIG. 4A-FIG. 4F show the intracellular uptake in SKBR3 human breast cancer cells of passive QDs (FIG. 4A), Herceptin-QDs+free 5-ALA (FIG. 4B), free 5-ALA (FIG. 4C), 5-ALA-QDs (FIG. 4D), 5-ALA-QDs+added free 5-ALA (FIG. 4E), and passive QDs+added free 5-ALA (FIG. 4F) at an incubation time of 3 hours at 5× magnification. The intracellular uptake in SKBR3 human breast cancer cells of passive QDs (FIG. 4G), Herceptin-QDs+free 5-ALA (FIG. 4H), free 5-ALA (FIG. 4I), 5-ALA-QDs (FIG. 4J), 5-ALA-QDs+added free 5-ALA (FIG. 4K), and passive QDs+added free 5-ALA (FIG. 4L) at an incubation time of 3 hours also shown at 100× magnification.

As shown in FIG. 2A-FIG. 2F through FIG. 4A-FIG. 4F, the intracellular uptake of QDs is enhanced by conjugating the QD to 5-ALA. Compare FIG. 2A and FIG. 2D and FIG. 3A and FIG. 3D. Without being bound by theory, the uptake enhancement is likely due to the active 5-ALA membrane transporters.

Surprisingly it was found that emission at 630 nm is at a maximum when both QD-5-ALA conjugates are co-administered with free 5-ALA (non-endogenous). Compare panels E of FIGS. 2, 3 and 4 with panels A-D and F of FIGS. 2, 3, and 4. The 5-ALA alone (panel C of FIGS. 2, 3 and 4), passive QDs (panel A of FIGS. 2, 3 and 4), or 5-ALA-QD alone without additional free 5-ALA (panel D of FIGS. 2, 3 and 4) all show weaker emission at 630 nm as compared with QD-5-ALA conjugates co-administered with free 5-ALA (non-endogenous) (panels E of FIGS. 2, 3, and 4). Without being bound by theory, it appears that endogenous free 5-ALA is limited and additional free 5-ALA is required for the augmented synthesis of PpIX.

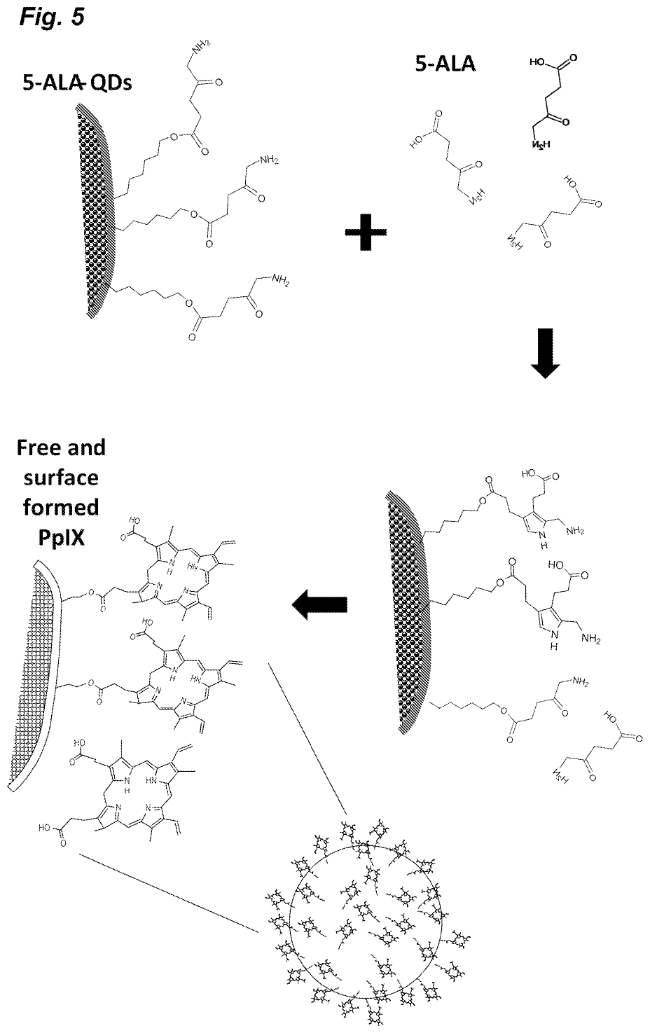

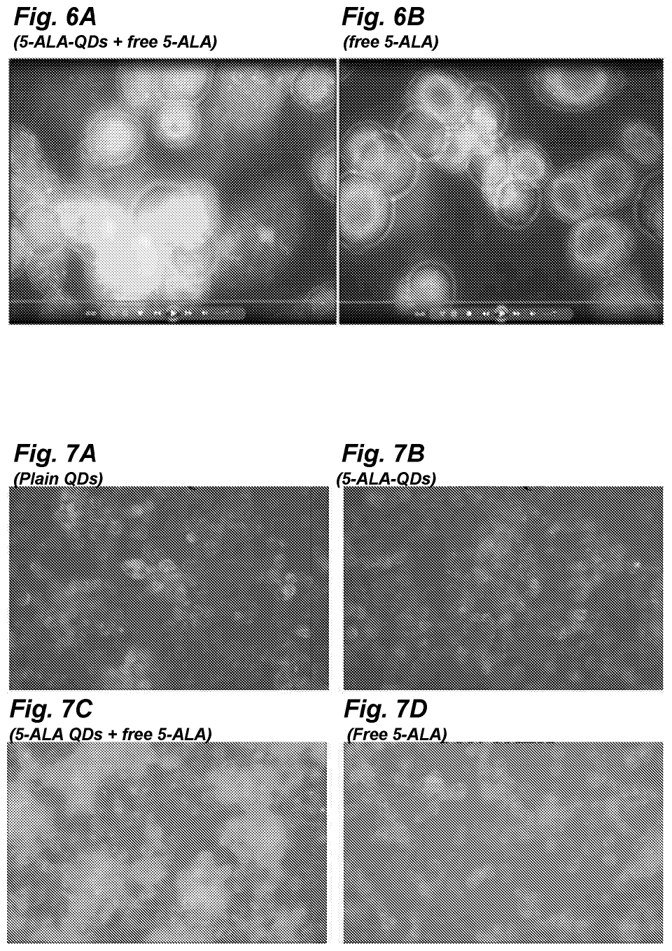

The results are surprising, and show that there is a mutually synergistic effect in the co-administration of QD-5-ALA conjugates with free 5-ALA (non-endogenous). Without being bound by theory, the synergistic effect likely results from the synthesis of PpIX taking place on the surface of the particle, as shown figuratively in FIG. 5. While free 5-ALA treatment shows rapid photo-bleaching and signal disappearance, the QD-5-ALA conjugates with free 5-ALA combination show continued strong emission. Compare panels E of FIGS. 2, 3, and 4 and FIG. 6A with FIG. 6B.

FIG. 6A and FIG. 6B demonstrate the observed resistance to photo-bleaching of 5-ALA-QD+free 5-ALA (FIG. 6A) versus free 5-ALA (FIG. 6B) after uptake by SKBR3 human breast cancer cells and after 1 minute of irradiation.

In addition to SKBR3 human breast cancer cells, the enhancement of 5-ALA-QD uptake by addition of free 5-ALA was also observed in several other cancer cell types. FIG. 7A-FIG. 7D compare detection of melanoma human A375 cancer cells after 5 hrs treatment with plain QDs (FIG. 7A), 5-ALA-QDs (50 μg/mL) (FIG. 7B), 5-ALA-QDs (50 μg/mL)+free 5-ALA (0.5 mM) (FIG. 7C), and 5-ALA alone (0.5 mM) (FIG. 7D).

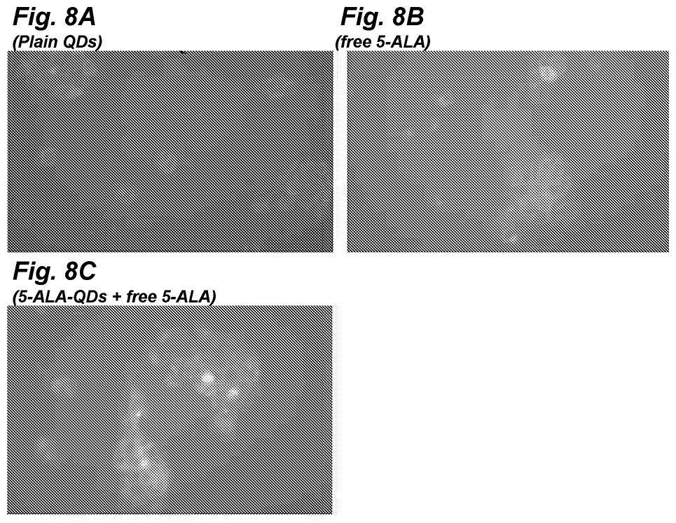

FIG. 8A-FIG. 8C compare detection of human squamous cell carcinoma A431 after 5 hrs treatment with plain QDs (FIG. 8A), 5-ALA alone (0.5 mM) (FIG. 8B), and 5-ALA-QDs (50 μg/mL)+free 5-ALA (0.5 mM) (FIG. 8C).

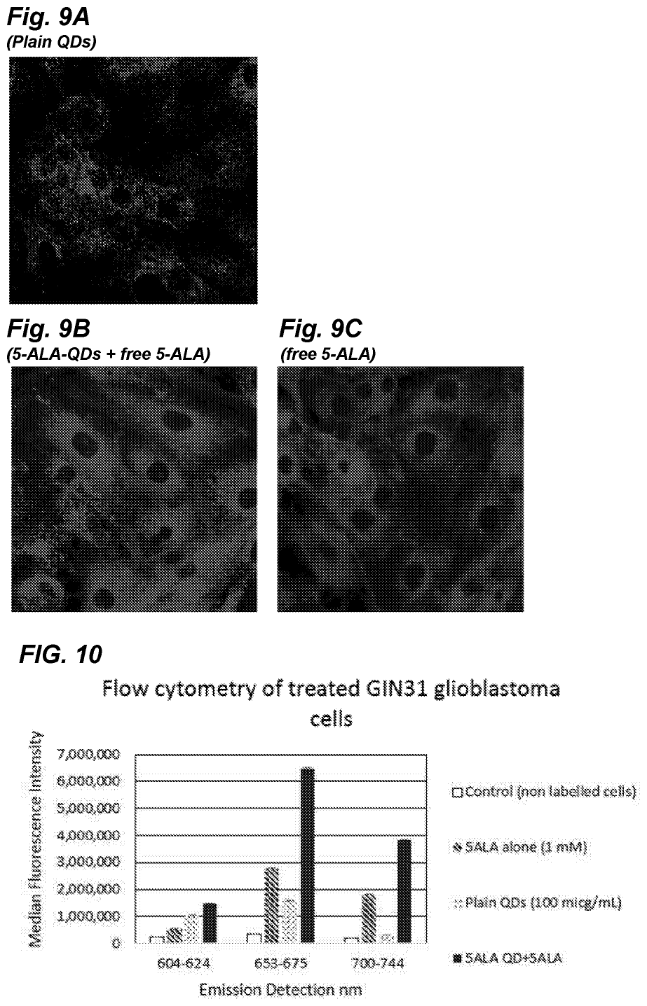

FIG. 9A-FIG. 9C compare confocal microscopic detection of human brain cancer cells GIN3 glioblastoma after 5 hrs treatment with plain QD (FIG. 9A), 5-ALA-QDs ((100 μg/mL)+5-ALA (1 mM) (FIG. 9B), and free 5-ALA (1 mM) (FIG. 9C).

FIG. 10 shows the results of flow cytometry of human brain cancer cells GIN3 glioblastoma after 5 hrs treatment with plain QD (100 μg/mL), 5-ALA-QDs (100 μg/mL)+5-ALA (1 mM), and free 5-ALA (1 mM) in comparison with control untreated and unlabeled cells.

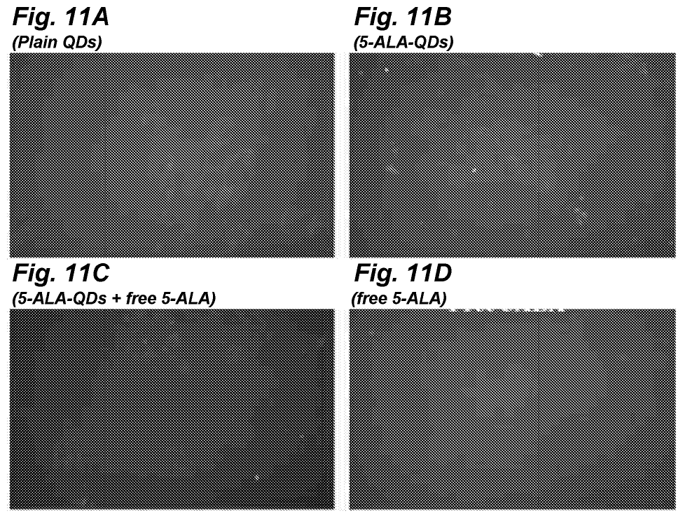

FIG. 11A-FIG. 11D show the lack of labeling of normal human fibroblast cells (HFF) after 16 hrs of treatment with plain QD (50 μg/mL) (FIG. 11A), 5-ALA-QDs (50 μg/mL) (FIG. 11B), 5-ALA-QDs (50 μg/mL)+free 5-ALA (0.2 mM) (FIG. 11C), and free 5-ALA alone (0.2 mM) (FIG. 11D).

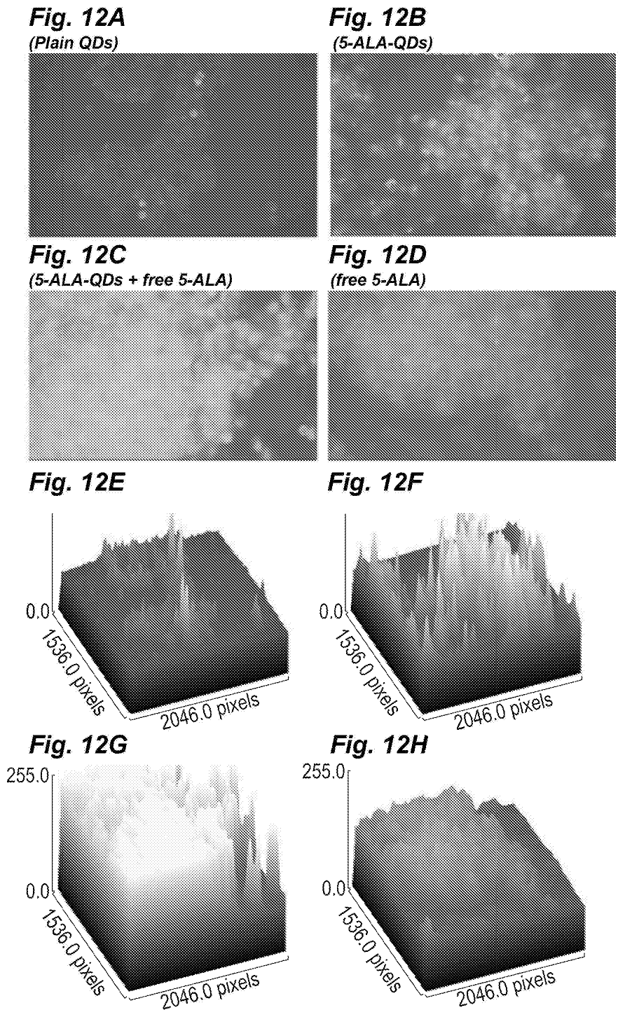

FIG. 12A-FIG. 12H show labelling of 3D cultured human pancreatic Mia-Paca-2 cancer (spheroids) after 16 hrs treatment with plain QD (50 μg/mL) (FIG. 12A and FIG. 12E), 5-ALA-QDs (50 μg/mL) (FIG. 12B and FIG. 12F), 5-ALA-QDs (50 μg/mL)+free 5-ALA (0.2 mM) (FIG. 12C and FIG. 12G), and free 5-ALA alone (0.2 mM) (FIG. 12D and FIG. 12H). ImageJ 1.51w software was used to generate surface plots of the red channel intensity from each image as shown in the lower panels FIG. 12E-FIG. 12H.

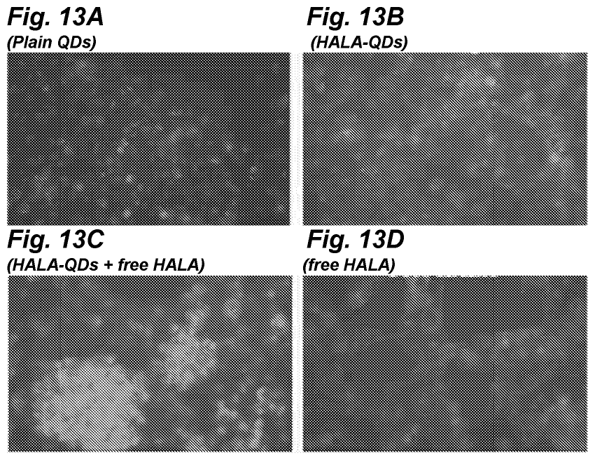

FIG. 13A-FIG. 13D show the similar performance of QD conjugates to derivatives of 5-ALA (i.e. a hexyl ester of 5 amino levulenic acid (HALA) by treating cultured human pancreatic Mia-Paca-2 cancer for 5 hrs with plain QD (FIG. 13A), HALA-QDs conjugate (50 μg/mL)(FIG. 13B), HALA-QDs conjugate (50 μg/mL)+free HALA (0.5 mM) (FIG. 13C), or free HALA alone (0.5 mM) (FIG. 13D). 5-Aminolevulinic acid hexyl ester hydrochloride is also known as hexaminolevulinate hydrochloride and has the following structure:

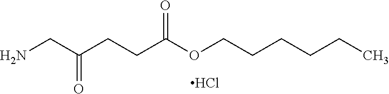

In certain embodiments, other esters of 5-amino levulenic acid are conjugated to QD including 5-ALA hexyl ester, 5-ALA methyl ester, aliphatic alcohol 5-ALA esters, glycoside ALA esters including α-glucose, α-mannose, or β-galactose esters of 5-ALA, and alkyl esters of 5-ALA.

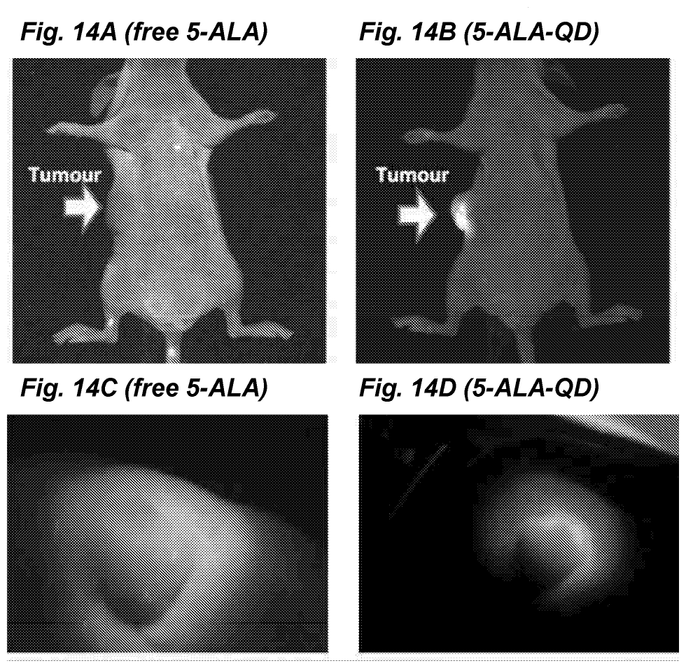

FIG. 14A-FIG. 14D show in vivo imaging of Mia Paca-2 human pancreatic tumors grown on the flank of immunosuppressed nude mice and then treated by intra-tumoral injection of 250 mg/Kg of 5-ALA (FIG. 14A and FIG. 14C) or 20 mg/Kg 5-ALA conjugated QDs dimensioned for emission at 630 nm (FIG. 14B and FIG. 14D). Imaging was performed on live animals after 5 h post injection using a Bruker In-vivoMS FXPRO whole animal imager at 410 nm excitation and 600 nm emission capture (FIG. 14A and FIG. 14B) or a hand-held blue flash light and a digital camera (FIG. 14C and FIG. 14D).

Without being bound by theory, the enhancement mechanism may be due to 1) increased uptake of the QDs by 5-ALA transporters; 2) surface synthesis and increased proximity of the formed PpIX to the QD particle, resulting in increased overall molecular extinction coefficient at the excitation wavelength; 3) co-localization of QDs and 5-ALA synthesis sites (e.g., mitochondria); 4) increased entrapment of synthesized PpIX due to the synthesis on surface of the particle; and/or 5) particle induced inhibition of PpIX conversion to heme.

Use of the Quantum Dot 5-ALA Conjugates:

The delivery of 5-ALA-QD or 5-ALA ester-QD conjugates with co-administration of free 5-ALA or 5-ALA ester can be used for multiple purposes. First, the 5-ALA-QD or 5-ALA ester-QD conjugates with co-administration of free 5-ALA or 5-ALA ester can be used for photodynamic therapy (PDT). When 5-ALA or 5-ALA ester converts to PpIX intracellularly and physically excited, for example, by photons, it produces reactive oxygen species (ROS), which lead to cell death. Previously, 5-ALA was prone to photo-bleaching and signal disappearance that the ROS pathway was insufficiently activated. For example, as discussed above, certain tumor types like those of the brain could not be treated by PDT using 5-ALA. With co-administration of 5-ALA-QD or 5-ALA ester-QD conjugates with co-administration of free 5-ALA or 5-ALA ester, photobleaching is reduced and the embodiments disclosed can be used to enhance the efficacy of 5-ALA PDT treatment by sustaining the signal emission, and therefore the ROS pathway, to treat tumor types that were not readily susceptible to 5-ALA PDT, and to better treat those tumor types that were previously treated with 5-ALA PDT in a more efficacious way. Increased ROS-pathway activation leads to cell death, which leads to tumor reduction and/or elimination. The presently disclosed invention provides for treatment and imaging of various abnormal proliferative tissue growths, cancers and precancerous conditions, such as, for example, cancers like gliomas, bladder cancer, melanomas, esophageal cancer, endobronchial cancer, cancers of the lung, bladder, prostate, bile duct, stomach, mouth, throat, larynx, cervix, vagina, and vulva. In certain embodiments, the PDT with 5-ALA-QD or 5-ALA ester-QD conjugates with co-administration of free 5-ALA or 5-ALA ester disclosed herein is utilized in treatment of cancerous skin cancers including melanoma, basal and squamous cell carcinoma and in treatment of precancerous lesions of the skin (including actinic keratosis). The presently disclosed improved PDT is also expected to find application to current off-label uses of PDT including for treatment of malignant pleural mesothelioma and mycosis fungoides. In certain embodiments proliferative inflammatory diseases of the skin and gastrointestinal tract are treated using the PDT with 5-ALA-QD or 5-ALA ester-QD conjugates with co-administration of free 5-ALA or 5-ALA ester as disclosed herein.

Currently PDT is FDA approved with administration of Porfimer sodium (PHOTOFRIN®) in treatment of esophageal cancer or Barrett's esophagus followed by passage of a fiber-optic strand down the throat through an endoscope for irradiation of the tissue. Currently PDT is also FDA approved with administration of Porfimer sodium (PHOTOFRIN®) for lung cancer treatments where imaging and irradiation is provided though a bronchoscope. The presently disclosed improvements provide such therapy using treatment with 5-ALA-QD or 5-ALA ester-QD conjugates with co-administration of free 5-ALA or 5-ALA ester. The combination of 5-ALA-QD or 5-ALA ester-QD conjugates with co-administered free 5-ALA or 5-ALA ester results in greatly augmented uptake of the QD with increased conversion to PpIX and reduced photobleaching making the combination suitable to treat a number of diseases that were not effectively treated with preexisting PDT. The QDs disclosed herein are in certain embodiments conjugated to tumor-specific ligands in addition to 5-ALA for tumor-specific targeting of the 5-ALA-QD or 5-ALA ester-QD conjugates and targeted administration of 5-ALA in accordance with the embodiments disclosed herein.

Second, because the 5-ALA-QD or 5-ALA ester-QD conjugates with co-administration of free 5-ALA or 5-ALA ester disclosed herein result in greater uptake of the QD and greater generation of detectable fluorescent PpIX intracellularly and are able to sustain a higher emission for a longer period of time, they can be used to as a marker (or label) in fluorescence guided surgery, treatment and imaging of various abnormal proliferative tissue growths, cancers and precancerous conditions previously listed.

Third, the administration of 5-ALA-QD or 5-ALA ester-QD conjugates with co-administration of free 5-ALA or 5-ALA ester embodiments disclosed herein can be used to screen tumors and inflammatory tissue samples to determine whether the tumor type of inflammatory tissue type would be susceptible to 5-ALA PDT. In one embodiment a screening kit is provided including one or more of 5-ALA-QD, 5-ALA hexyl ester-QD, 5-ALA methyl ester-QD, aliphatic alcohol 5-ALA ester-QD, glycoside 5-ALA ester-QD and 5-ALA alkyl ester-QD together with one or more of free 5-ALA, free 5-ALA hexyl ester, free 5-ALA methyl ester, free aliphatic alcohol 5-ALA ester, free glycoside 5-ALA ester and free 5-ALA alkyl ester. Examples of glycoside 5-ALA esters include α-glucose, α-mannose, or β-galactose esters of 5-ALA. The screening kit is used to establish and select the best type of 5-ALA-QD and free 5-ALA depending on the cell type being treated and the 5-ALA uptake mechanism most active in the particular cell being treated. The 5-ALA-QD or 5-ALA ester-QD conjugates with co-administration of free 5-ALA or 5-ALA ester embodiments disclosed herein may be provided as an analytical kit that can be sold as a pre-mixed composition to screen tissues, tumors (or biopsies thereof) to determine the susceptibility of the tested tumor to 5-ALA treatment.

The administration of the 5-ALA-QD together with free 5-ALA in embodiments disclosed herein can be enteral or parenteral. For example, the 5-ALA-QD and free 5-ALA can be administered subcutaneously, intravenously, intramuscular, topically, and orally in various embodiments. Examples include bolus injection or IV infusions. For administration, the 5-ALA-QD or 5-ALA ester-QD conjugates with co-administration of free 5-ALA or 5-ALA ester will be applied to the tissue being treated and allowed to become internalized into cells for a period to time sufficient to allow for conversion to PpIX prior to irradiation. This period will be determined empirically depending on the tissue being treated. In certain embodiments, the 5-ALA-QD or 5-ALA ester-QD conjugates with co-administration of free 5-ALA or 5-ALA ester will be administered 10 to 20 hours prior to irradiation. In certain embodiments the drug combination of 5-ALA-QD or 5-ALA ester-QD conjugate with co-administration of free 5-ALA or 5-ALA ester will be administered 14 to 18 hours prior to irradiation.

The 5-ALA-QD or 5-ALA ester-QD conjugates and free 5-ALA embodiments disclosed herein can come as a pre-mixed composition that can be readily administered to a patient in need thereof.

In one embodiment, the 5-ALA-QD or 5-ALA ester-QD conjugate is formulated with 5-ALA or the corresponding 5-ALA ester and packaged in a unit dose volume to be administered in a single procedure, such as through the skin and into a tumor tissue. In one embodiment, the volume per unit dose is determined on the basis of the anatomy of the administration site as well as the desired distribution area.

These and other advantages of the present invention will be apparent to those skilled in the art from the foregoing specification. Accordingly, it is to be recognized by those skilled in the art that changes or modifications may be made to the above-described embodiments without departing from the broad inventive concepts of the invention. It is to be understood that this invention is not limited to the particular embodiments described herein, but is intended to include all changes and modifications that are within the scope and spirit of the invention.

1. A method for the enhancement of intracellular PpIX fluorescence comprising:
administering quantum dots (QDs) conjugated to 5-Aminolevulinic acid (5-ALA) or esters of 5-ALA to a tissue;
co-administering free 5-ALA or 5-ALA esters to the tissue;
allowing the QD-5-ALA or QD-5-ALA ester conjugates the co-administered free 5-ALA or 5-ALA esters to be internalized by cells within the tissue and form intracellular PpIX; and
physically exciting the QD-5-ALA or QD-5-ALA ester conjugates to induce PpIX fluorescence.

2. (canceled)

3. The method of claim 1, wherein the step of physically exciting QD-5-ALA or QD-5-ALA ester conjugates comprises irradiation.

4. The method of claim 1, wherein the induced intracellular PpIX fluorescence is utilized for (i) photodynamic therapy of abnormal tissue proliferation, pre-neoplastic tissues, neoplastic tissues and reduction in tumor size or labelling of tumors, or (ii) labelling and visualization of abnormal tissue proliferation, pre-neoplastic tissues, and neoplastic tissues.

5. (canceled)

6. (canceled)

7. The method of claim 1, wherein the administration is used to screen tissues that may be susceptible to 5-ALA or 5-ALA ester treatment.

8. The method of claim 1, wherein the esters of 5-ALA are selected from 5-ALA hexyl esters, 5-ALA methyl esters, aliphatic alcohol 5-ALA esters, glycoside 5-ALA esters including α-glucose, α-mannose, or β-galactose esters of 5-ALA, and alkyl esters of 5-ALA.

9. The method of claim 1, wherein QDs are further conjugated to at least one tumor-specific ligand, wherein the tumor specific ligand is specific for EGFR, PD-L1, PD-L2, HER2, CEA, CA19-9, CA125, telomerase proteins and subunits, CD20, CD25, CD30, CD33, CD52, CD73, CD109, VEGF-A, CTLA-4, or RANK ligand.

10. (canceled)

11. The method of claim 1, wherein the QDs (i) further comprise a polymerizable ligand that results in QD aggregation upon physical excitation of the QDs, or (ii) further include a cellular uptake enhancer, a tissue penetration enhancer, or any combination thereof;
wherein the cellular uptake enhancer is selected from one or more of trans-activating transcriptional activators (TAT), Arg-Gly-Asp (RGD) tri-peptides, linear and cyclic peptides including the RGD motif, or poly arginine peptides; and
wherein the tissue penetration enhancer is selected from one or more of saponins, cationic lipids, and Streptolysin O (SLO).

12. (canceled)

13. (canceled)

14. (canceled)

15. A method for facilitating cell death comprising:
administering quantum dots (QDs) conjugated to 5-Aminolevulinic acid (5-ALA) or esters thereof to undesired cells;
co-administering free 5-ALA or esters thereof to the undesired cells; and
physically exciting the QDs conjugated to 5-ALA or esters thereof to induce PpIX fluorescence and generation of reactive oxygen species (ROS) that facilitate cell death.

16. The method according to claim 15, wherein the undesired cells are precancerous cells, tumor cells, or inflammatory tissue and the method is performed in vivo.

17. An analytical kit for determining improved cellular uptake of quantum dots (QDs) conjugated to 5-Aminolevulinic acid (5-ALA) or esters thereof comprising:
QDs conjugated to 5-Aminolevulinic acid (5-ALA) or esters thereof; and
additional free unconjugated 5-ALA or esters thereof for co-administration with the QDs conjugated to 5-ALA or esters thereof.

18. The analytical kit of claim 17, wherein the kit includes a panel of 5-ALA and 5-ALA ester conjugates to QD, the panel including at least two of the group consisting of 5-ALA-QD, 5-ALA hexyl ester-QD, 5-ALA methyl ester-QD, aliphatic alcohol 5-ALA ester-QD, glycoside 5-ALA ester-QD and 5-ALA alkyl ester-QD together with one or more of additional free 5-ALA, free 5-ALA hexyl ester, free 5-ALA methyl ester, free aliphatic alcohol 5-ALA ester, free glycoside 5-ALA ester and free 5-ALA alkyl ester.

19. The analytical kit of claim 17, wherein the glycoside 5-ALA esters are selected from α-glucose, α-mannose, and β-galactose esters of 5-ALA.

20. A composition comprising quantum dots (QDs) conjugated to 5-Aminolevulinic acid (5-ALA) or esters of 5-ALA in co-formulation with additional free unconjugated 5-ALA or esters thereof.

21. (canceled)

22. The composition of claim 20, wherein the esters of 5-ALA are selected from 5-ALA hexyl ester, 5-ALA methyl ester, aliphatic alcohol 5-ALA esters, glycoside 5-ALA esters including α-glucose, α-mannose, or β-galactose esters of 5-ALA, and alkyl esters of 5-ALA.

23. The composition of claim 20, wherein the QDs are further conjugated to at least one tumor-specific ligand.

24. The composition of claim 23, wherein the tumor specific ligand is specific for EGFR, PD-L1, PD-L2, HER2, CEA, CA19-9, CA125, telomerase proteins and subunits, CD20, CD25, CD30, CD33, CD52, CD73, CD109, VEGF-A, CTLA-4, or RANK ligand.

25. The composition of claim 20, wherein the QDs further include a polymerizable ligand that results in QD aggregation upon physical excitation of the QD.

26. The composition of claim 20, wherein the QDs further include a cellular uptake enhancer, a tissue penetration enhancer, or any combination thereof.

27. The composition of claim 26, wherein the cellular uptake enhancer is selected from one or more of trans-activating transcriptional activators (TAT), Arg-Gly-Asp (RGD) tri-peptides, linear and cyclic peptides including the RGD motif, or poly arginine peptides.

28. The composition of claim 26, wherein the tissue penetration enhancer is selected from one or more of saponins, cationic lipids, and Streptolysin O (SLO).
